(19) United States
(12) Patent Application Publication
(10) Pub. No.: US 2021/0311075 A1
(43) Pub. Date: Oct. 7, 2021

(54) METHODS FOR DETECTING DISORDERS RELATED TO CALCIUM DISCHARGE FROM INTRACELLULAR STORES

(71) Applicant: The Regents of the University of California, Oakland, CA (US)

(72) Inventor: Alla F. FOMINA, Davis, CA (US)

(73) Assignee: The Regents of the University of California, Oakland, CA (US)

(21) Appl. No.: 16/776,396

(22) Filed: Jan. 29, 2020

Related U.S. Application Data
(63) Continuation of application No. PCT/US2018/044346, filed on Jul. 30, 2018.
(60) Provisional application No. 62/539,354, filed on Jul. 31, 2017.

(51) Int. Cl. G01N 33/68; G01N 33/569

(52) U.S. Cl. CPC G01N 33/6872; G01N 33/56972; G01N 2800/50; G01N 2800/40; G01N 2800/2821

(57) ABSTRACT

Provided herein are methods for determining whether a subject has an increased likelihood of having a disorder related to abnormal intracellular calcium store discharge. In some embodiments, the method is useful for determining whether the subject has an increased likelihood of having a neurodegenerative disease, skeletal muscle disease, cardiac muscle disease, autoimmune disease, cancer, diabetes, or has been exposed to an environmental pollutant. Kits are also provided herein.

### CROSS-REFERENCE TO RELATED APPLICATIONS

This application is a continuation of International Application No. PCT/US2018/044346, filed Jul. 30, 2018, which claims priority to U.S. Provisional Application No. 62/539,354, filed Jul. 31, 2017, the disclosures of which are herein incorporated by reference in their entirety for all purposes.

### BACKGROUND OF THE INVENTION

A number of human diseases, including but not limited to Alzheimer's disease (23), heart failure (24), muscle fatigue, Duchenne muscular dystrophy (25), malignant hyperthermia, cancer (26), and diabetes (27) are associated with mutations and/or post-translational modifications in proteins that regulate levels of intracellular free calcium, through regulating the calcium discharge from the intracellular calcium stores. Although genetic analyses are available to identify disease-related mutations, these methods report the presence of a risk factor, not the presence or stage of the disease itself. Moreover, aberrations in calcium handling in different types of cells from affected individuals appear prior to the onset of clinical symptoms.

Worldwide, nearly 44 million people have Alzheimer's disease (AD; www.alzheimers.net). AD afflicts an estimated 5.2 million people in the U.S., with an expected increase to 13.8-16 million by the year 2050 (www.alz.org). Currently, diagnosis of AD relies largely on documenting mental decline after severe brain damage has already occurred. Thus, there has been a need to discover an easy and accurate way to detect AD before the devastating symptoms begin. There are currently no validated biomarkers for AD but the most promising approaches include analyses of beta-amyloid and tau levels in cerebrospinal fluid or other parts of the body (e.g., eye), brain changes detectable by imaging, and genetic risk profiling. These approaches are either invasive (e.g., samples are taken from cerebrospinal fluid or the eye); expensive (e.g., brain imaging), or have low diagnostic or predictive power (e.g., genetic analysis). Thus, there has been an unmet need to develop new minimally invasive biomarker-based diagnostic methods that will allow early preclinical detection of AD.

Point mutations in the ryanodine receptor type 1 (RyR1) and associated proteins result in increased in calcium discharge from the skeletal muscle sarcoplasmic reticulum and, consequently, several clinically distinct human congenital muscle diseases, including malignant hyperthermia (MH), central core disease (CCD), and specific variants of multi-minicore disease (MmD) and nemaline rod myopathy (NM) (4, 5). There is an association between CCD and MH, as individuals with MH may have central cores on muscle biopsy, and CCD patients are prone to MH episodes. In addition, heat intolerance and exercise and stress-induced muscle syndromes are associated with RyR1 defects (6). At least 30 mutations have been identified in the RYR1 gene and validated as causal for MH susceptibility. The recently estimated prevalence of genetic abnormalities associated with MH is 1:400 individuals. The discordance between MH diagnosed by the presence of causative mutations and skeletal muscle contracture tests (a functional test establishing the presence of MH reactivity) has been reported (8). Establishing a MH-positive or MH-negative diagnosis has clinical significance in terms of subsequent anesthetic treatment of a patient and his/her family members.

Currently, all tests that can produce definitive diagnoses of RyR1 dysfunction-related diseases require skeletal muscle biopsy, an invasive procedure performed under local or general anesthesia and, in many cases, in young children <5 years of age (9). These tests must be completed within five hours of biopsy at a certified MH diagnostic center. Because of the invasive surgery, as well as procedural and logistic complexities, the costs of the tests are high, and only about 10% of all patients who are eligible to undergo testing for RyR1 dysfunction are actually tested.

A variety of minimally invasive diagnostic tests, which are in development at present, include nuclear magnetic resonance spectroscopy to evaluate ATP depletion during graded exercise in vivo, and measuring levels of adenosine and inosine produced by peripheral blood-derived B lymphocytes challenged with RyR1 agonists (10). The common limitations of these tests are that they all measure levels of chemicals affected by a variety of cellular reactions and, therefore, display strong intrinsic variability, require expensive and sophisticated equipment, and a team of technicians experienced in performing these tests and interpreting the results.

Accordingly, there is a need in the art for new minimally invasive (e.g., can be performed on an easily-obtainable sample such as a blood sample), sensitive, and inexpensive (e.g., not requiring expensive equipment or chemicals) methods for the detection of intracellular calcium handling dysfunction (e.g., RyR1 and/or inositol trisphosphate receptor dysfunction) and the preclinical detection of diseases—such as AD, MH, Duchenne muscular dystrophy, heart failure, muscle fatigue, cancer, and diabetes—that arise from abnormal discharge from intracellular calcium stores. The present invention satisfies this need, and provides related advantages as well.

### BRIEF SUMMARY OF THE INVENTION

In one aspect, the present invention provides a method for determining whether a subject has an increased likelihood of having a disorder related to abnormal intracellular calcium store discharge, the method comprising:

(a) detecting an intracellular calcium concentration F₂ in a lymphocyte obtained from the subject while the lymphocyte is in contact with an antagonist of calcium influx;
(b)(1) detecting an intracellular calcium concentration F₁ in the lymphocyte while the lymphocyte is in contact with a calcium-containing solution, and/or
(b)(2) detecting an intracellular calcium concentration F₃ in the lymphocyte after the lymphocyte has been contacted with a calcium-mobilizing agent, or before and after the lymphocyte has been contacted with the calcium-mobilizing agent;
(c)(1) determining an intracellular calcium concentration difference Δ₁, wherein Δ₁ equals F₁ minus F₂, and/or
(c)(2) determining an intracellular calcium concentration difference Δ₂, wherein Δ₂ equals F₃ minus F₂; and
(d) determining whether the subject has an increased likelihood of having a disorder related to abnormal intracellular calcium store discharge based upon the value of Δ₁ and/or Δ₂.

In some embodiments, different lymphocytes are contacted in steps (a), (b)(1), and/or (b)(2). In some embodiments, the same lymphocyte is contacted in all of steps (a), (b)(1), and/or (b)(2). In some embodiments, step (b)(1) is performed before step (a). In some embodiments, step (b)(2) is performed after step (a).

In some embodiments, the lymphocyte is isolated from a sample obtained from the subject. In some embodiments, the sample is a whole blood sample, lymph sample, tonsillar tissue sample, lung tissue sample, mucosal tissue sample, or a combination thereof. In some embodiments, the lymphocyte is a T lymphocyte. In some embodiments, the lymphocyte comprises a plurality of lymphocytes.

In some embodiments, the lymphocyte is contacted with the calcium-mobilizing agent while the lymphocyte is in contact with the antagonist of calcium influx. In some embodiments, F₂ is the lowest intracellular calcium concentration detected before or after the lymphocyte is contacted with the calcium-mobilizing agent. In some embodiments, F₃ is detected between about 1 second and about 10 minutes after the lymphocyte has been contacted with the calcium-mobilizing agent. In some embodiments, F₃ is the highest intracellular calcium concentration detected after the lymphocyte has been contacted with the calcium-mobilizing agent.

In some embodiments, F₃ is detected at two or more time points, wherein the earliest time point is t₁ and the latest time point is t₂. In some embodiments, t₁ is about 5 to 10 minutes before the lymphocyte is contacted with the calcium-mobilizing agent. In some embodiments, t₂ is about 5 to 10 minutes after the lymphocyte has been contacted with the calcium-mobilizing agent.

In some embodiments, the calcium-containing solution comprises a physiological saline solution such as Ringer's solution, Tyrode's solution, Hank's solution, or a combination thereof. In some embodiments, the concentration of calcium in the calcium-containing solution is between about 0.1 mM and about 100 mM. In some embodiments, the concentration of calcium in the calcium-containing solution is about 2 mM.

In some embodiments, the antagonist of calcium influx comprises a calcium-free physiological saline solution, a calcium chelating agent, a calcium channel blocker, or a combination thereof. In some embodiments, the calcium chelating agent is selected from the group consisting of ethylene glycol-bis(O-aminoethyl ether)-N,N,N′,N′-tetraacetic acid (EGTA), ethylenediaminetetraacetic acid (EDTA), 1,2-bis(o-aminophenoxy)ethane-N,N,N′,N′-tetraacetic acid (BAPTA), N-carboxymethyl-N′-(2-hydroxyethyl)-N,N′-ethylenediglycine (HEDTA, also known as HEEDTA), pentetic acid, diethylenetriaminepentaacetic acid (DTPA), and a combination thereof.

In some embodiments, the calcium channel blocker is selected from the group consisting of a store-operated calcium channel blocker, a transient receptor potential (TRP) channel blocker, and a combination thereof. In some embodiments, the store-operated calcium channel blocker is selected from the group consisting of La³⁺, Gd³⁺, 2,6-difluoro-N-(1-(2-phenoxybenzyl)-1H-pyrazol-3-yl)benzamide (GSK-5503A), an imidazole, 2-aminoethoxydiphenylborate (2-APB), capsaicin, 5-nitro-2-(3-phenylpropylamino)-benzoic acid (NPPB), a bistrifluoromethyl-pyrazole derivative, diethylstilbestrol (DES), bromenol lactone (BEL), a bile acid, (1-(5-chloronaphthalene-1-sulfonyl)homopiperazine (ML-9), 4-(N-[[(6-hydroxy-1-naphthalenyl)amino]thioxomethyl]-2-furancarboxamide (CPD5J), 1-(5-chloronaphthalenesulfonyl)homopiperazine hydrochloride, (N-propylargylnitrendipine (MRS 1845), (1-[2-(4-Methoxyphenyl)-2-[3-(4-methoxyphenyl)propoxy]ethyl-1H-imidazole hydrochloride (SKF 96365 HCl), N-[4-[3,5-bis(trifluoromethyl)-1H-pyrazol-1-yl]phenyl]-4-methyl-1,2,3-thiadiazole-5-carboxamide (YM 58483), 2,6-difluoro-N-(1-(4-hydroxy-2-(trifluoromethyl)benzyl)-1H-pyrazol-3-yl)benzamide (GSK-7975A), N-[4-[3,5-bis(trifluoromethyl)-1H-pyrazol-1-yl]phenyl]-3-fluoro-4-pyridinecarboxamide (Pyr6), and a combination thereof.

In some embodiments, the TRP channel blocker is selected from the group consisting of La³⁺, Gd³+, 2-[[3-(trifluoromethyl)phenyl]amino]benzoic acid (flufenamic acid), 1-[(4-chlorophenyl)methyl]-2-(1-pyrrolidinylmethyl)-1H-benzimidazole hydrochloride (clemizole hydrochloride), N-butyl-1H-benzimidazol-2-amine hydrochloride (M 084 hydrochloride), 3-([1,4′-bipiperidin]-1′-ylmethyl)-7-bromo-N-(1-phenylcyclopropyl)-2-[3-(trifluoromethyl)phenyl]-4-quinolinecarboxamide (GSK 2193874), 1,2,3,6-tetrahydro-1,3-dimethyl-N-[4-(1-methylethyl)phenyl]-2,6-dioxo-7H-purine-7-acetamide (HC-030031), N-(2-aminoethyl)-N-(4-(benzyloxy)-3-methoxybenzyl)thiophene-2-carboxamide hydrochloride (M8-B), a mefenamic acid analog, 2-(3-methylphenyl)aminobenzoic acid (3-MFA), 4-methyl-2-(1-piperidinyl)-quinoline (ML204), 3-[[[(1R)-1-(4-fluorophenyl)ethyl](3-quinolinylcarbonyl)amino]methyl]-benzoic acid (PF-05105679), N-[4-[3,5-bis(trifluoromethyl)-1H-pyrazol-1-yl]phenyl]-4-methyl-benzenesulfonamide (Pyr10), N-(4-(4-isopropylpiperazin-1-ylsulfonyl)phenyl)-2-nitro-4-(trifluoromethyl)benzamide (RN-9893), 4-(N-(3-chloro-5-(trifluoromethyl)pyridin-2-yl)-N-(4-(trifluoromethoxy)benzyl)sulfamoyl)benzoic acid (RQ-00203078), GsMTx4, 1-[2-(4-methoxyphenyl)-2-[3-(4-methoxyphenyl)propoxy]ethyl-1H-imidazole hydrochloride (SKF 96365 hydrochloride), (1-[4-[(2,3,3-trichloro-1-oxo-2-propen-1-yl)amino]phenyl]-5-(trifluoromethyl)-1H-pyrazole-4-carboxylic acid) (Pyr3), 4-methyl-2-(1-piperidinyl)quinolone (ML 204), and a combination thereof.

In some embodiments, the calcium-mobilizing agent is selected from the group consisting of a sarco/endoplasmic reticulum Ca²⁺-ATPase (SERCA) inhibitor, a calcium ionophore, a membrane receptor agonist, an intracellular receptor agonist, an intracellular calcium chelator, a cell-permeable lipophilic ester thereof, a caged analog thereof, and a combination thereof. In some embodiments, the SERCA inhibitor is selected from the group consisting of thapsigargin, cyclopiazonic acid (CPA), 2,5-di(t-butyl)-1,4-hydroquinone (BHQ), nonylphenol, bisphenol, bisphenol A, tetrabromobisphenol A (TBBPA), 4-chloro-m-cresol, orthovanadate, a flavonoid, 2-aminoethoxydiphenyl borate (2APB), curcumin, paxilline, alisol B, mastoparan, peptide M391, ivermectin, cyclosporin A, rapamycin, chlorpromazine, calmidazolium, fluphenazine, diethylstilbestrol (DES), 1,3-dibromo-2,4,6-tris(methylisothiouronium) benzene, stable analog of HA 14-1 (sHA 14-1), a cell-permeable lipophilic ester thereof, a caged analog thereof, and a combination thereof. In some embodiments, the flavonoid is selected from the group consisting of 3,6-dihydroxyflavone, quercitin, galangin, a cell-permeable lipophilic ester thereof, a caged analog thereof, and a combination thereof. In some embodiments, the calcium-mobilizing agent is present at a concentration of about 0.01 μM to about 10 mM.

In some embodiments, the calcium ionophore is selected from the group consisting of ionomycin, A23187 (calcimycin), 4-bromo A23187, and a combination thereof. In some embodiments, the intracellular calcium chelator is selected from N,N,N′,N′-tetrakis(2-pyridylmethyl)ethane-1,2-diamine (TPEN), EGTA, EDTA, BAPTA, HEDTA, HEEDTA, pentetic acid, DTPA, a cell-permeable lipophilic ester thereof, a caged analog thereof, and a combination thereof. In some embodiments, the membrane receptor agonist is a T lymphocyte receptor agonist. In some embodiments, the intracellular receptor agonist is an inositol trisphosphate receptor (IP3R) agonist and/or a ryanodine receptor (RyR) agonist.

In some embodiments, the intracellular calcium concentration F₁, F₂, and/or F₃ is detected by observing or recording a signal. In some embodiments, the signal is proportional to the intracellular calcium concentration. In some embodiments, the signal is generated using a fluorescent calcium indicator. In some embodiments, the fluorescent calcium indicator is selected from the group consisting of fura-2, indo-1, fluo-3, fluo-4, calcium green-1, mag-fura-2, fura red, fura-FF, indo-1, mag-indo-1, mag-fluo-4, Oregon green 488 BAPTA-1, quin-2, rhod-2, a genetically-encoded sensor, an analog thereof, an acetoxymethyl ester thereof, an acetate ester thereof, a water soluble salt thereof, a dextran variant thereof, a cadaverine variant thereof, a thiol-reactive iodoacetamide thereof, and a combination thereof.

In some embodiments, the lymphocyte is loaded with the fluorescent calcium indicator before being contacted with the calcium-containing solution.

In some embodiments, the method further comprises contacting the lymphocyte with a lymphocyte activating agent. In some embodiments, the lymphocyte activating agent is selected from the group consisting of ionomycin, phorbol myristate acetate (PMA), phytohemagglutinin (PHA), lipopolysaccharide (LPS), concanavalin A (Con A), pokeweed mitogen (PWM), an anti-CD3 antibody, an anti-CD28 antibody, and a combination thereof.

In some embodiments, the determination of whether the subject has an increased likelihood of having a disorder related to abnormal intracellular calcium store discharge is based upon the difference between Δ₁ and Δ₂. In some embodiments, the determination of whether the subject has an increased likelihood of having a disorder related to abnormal intracellular calcium store discharge is based upon the ratio of Δ₁ to Δ₂. In some embodiments, the value of Δ₁, Δ₂, the difference between Δ₁ and Δ₂, or the ratio of Δ₁ to Δ₂ is compared to a reference value. In some embodiments, the reference value is determined from a control subject or a control population. the control subject or control population is a subject or a population of subjects who do not have a disorder related to abnormal intracellular calcium store discharge.

In some embodiments, the disorder related to abnormal intracellular calcium store discharge is selected from the group consisting of Alzheimer's disease (AD), Parkinson's disease, Huntington's disease, malignant hyperthermia (MH), central core disease, multi-minicore disease (MmD), nemaline rod myopathy (NM), a T lymphocyte-mediated autoimmune disease, and exposure to a chemical pollutant. In some embodiments, the chemical pollutant is selected from the group consisting of a polychlorinated biphenyl compound, triclosan, bisphenol A, and a combination thereof.

In some embodiments, the T lymphocyte-mediated autoimmune disease is selected from the group consisting of multiple sclerosis (MS), myasthenia gravis, an autoimmune neuropathy, uveitis, autoimmune hemolytic anemia, pernicious anemia, autoimmune thrombocytopenia, temporal arteritis, anti-phospholipid syndrome, autoimmune vasculitis, Bechet's disease, atherosclerosis, psoriasis, dermatitis herpetiformis, pemphigus vulgaris, vitiligo, mycosis fungoides, allergic contact dermatitis, atopic dermatitis, lichen planus, pityriasis lichenoides at varioliforms acute (PLEVA), Crohn's disease, ulcerative colitis, primary biliary cirrhosis, autoimmune hepatitis, type I diabetes mellitus, Addison's disease, Grave's disease, and Hashimoto's thyroiditis.

Other objects, features, and advantages of the present invention will be apparent to one of skill in the art from the following detailed description and figures.

### BRIEF DESCRIPTION OF THE DRAWINGS

FIG. 1 shows a schematic representation of the mechanisms of cytosolic calcium handling in T lymphocytes and changes in the fluorescence levels of a calcium indicator (F) in response to the removal of extracellular calcium (F₂) and the blockade of SERCA (F₃) relative to the basal intracellular calcium level (F₁) in a normal extracellular solution containing 2 mM Ca²⁺.

FIG. 2 shows the early onset of autoimmune disease in mice carrying the gain-of-function R163C RyR1 mutation. The graph depicts the cumulative clinical score in wild-type (WT) and R163C mice after immunization with MOG₃₅₋₅₅ peptide to induce experimental autoimmune encephalomyelitis (EAE). Mice were immunized on day 0 and scored daily using the following criteria: (1) tail weakness, (2) difficulty righting, (3) hind limb paralysis, (4) forelimb weakness or paralysis, or (5) moribund or dead. Each group contained four mice (p<0.01).

FIGS. 3A and 3B show intracellular calcium measurements in T lymphocytes. FIG. 3A shows changes in fura-2 fluorescence (expressed as the ratio of fluorescence at 340 nm and 380 nm excitation wavelengths; F₃₄₀/F₃₈₀), which is proportional to changes in the intracellular calcium concentration in T lymphocytes isolated from the wild-type (WT) control, 5×FAD, and R163C mice. Shown are average traces recorded from at least 20 cells in one representative experiment. FIG. 3B shows average values of the Δ₁/Δ₂ ratio in T lymphocytes isolated from WT, 5×FAD, and R163C mice, including R163C lymphocytes that were preincubated with the ryanodine receptor (RyR) blockers ryanodine (Ry) and dantrolene (Da). n=number of experiments.

### DETAILED DESCRIPTION OF THE INVENTION

### I. Introduction

The present invention is based, in part, on the discovery of a new method for detecting intracellular calcium dysregulation. Such dysregulation can be related to, for example, “leaky” endoplasmic reticulum (ER) or sarcoplasmic reticulum (SR) calcium stores, which can be caused by, for example, aberrant calcium store release through defective ryanodine receptors (RyRs) and/or inositol trisphosphate receptors (IP3Rs). A number of RyR mutations (e.g., the R163C mutation) have been implicated in intracellular calcium dysregulation. Furthermore, intracellular calcium dysregulation can be exacerbated by a disease and/or exposure to various chemical agents. According to the methods of the present invention, the intracellular calcium concentration in a peripheral blood mononuclear cell (PBMC), such as a lymphocyte (e.g., a T lymphocyte), is determined while the cell is in contact with an antagonist of calcium influx (the F₂ intracellular calcium concentration) and while the cell is in contact with a calcium-containing solution (the F₁ intracellular calcium concentration) and/or a calcium-mobilizing agent (the F₃ intracellular calcium concentration). A first intracellular calcium concentration difference (Δ₁) is taken as the difference between F₁ and F₂ (i.e., Δ₁=F₁− F₂) and/or a second intracellular calcium difference (Δ₂) is taken as the difference between F₃ and F₂ (i.e., Δ₂=F₃− F₂). By determining the value of Δ₁ and/or Δ₂, the inventors of the present invention have discovered that it is possible to detect dysregulation of intracellular calcium (e.g., due to “leaky” endoplasmic reticulum (ER) or sarcoplasmic reticulum (SR) calcium stores resulting from aberrant calcium release, for example, through RyRs or IP3Rs).

The present invention is also based on, in part, on the discovery that detecting intracellular calcium dysregulation according to the methods described herein is useful for determining whether a subject has an increased likelihood for having or developing any number of diseases that result from, at least in part, intracellular calcium dysregulation (e.g., Alzheimer's disease, malignant hyperthermia, central core disease, multi-minicore disease, nemaline rod myopathy, heart failure, muscle fatigue, Duchenne muscular dystrophy, cancer, diabetes, exposure to chemical pollutants, and T lymphocyte-mediated autoimmune diseases).

Accordingly, the present invention allows for improved diagnostic methods that are less invasive, less expensive, and faster than existing alternatives. The improved diagnostic methods can be used to detect diseases such as Alzheimer's disease while the subject is still in the preclinical stages, and can also be used to predict the responses of muscles to anesthetic agents or various environmental factors (e.g., exposure to chemical pollutants).

### II. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.”

The terms “subject,” “individual,” and “patient” are used interchangeably herein to refer to a vertebrate, preferably a mammal, more preferably a human. Mammals include, but are not limited to, rodents (e.g., mice, rats), simians, humans, farm animals, sport animals, and pets.

The term “lymphocyte” refers to a subtype of white blood cells that includes T lymphocytes (also called “T cells”), B lymphocytes (also called “B cells”), and natural killer (NK) cells.

T lymphocytes mature in the thymus and play a central role in cell-mediated immunity. T lymphocytes are distinguished from other types of lymphocytes by the presence of T-cell receptors that are present on the cell surface. T lymphocytes are subdivided into several types, including effector T cells, helper T cells, cytotoxic (killer) T cells, memory cells, regulatory (suppressor) T cells, and natural killer T cells. Effector T cells refer to a broad category that includes several T lymphocyte types (including helper, killer, and regulatory cells), and generally are cells that respond to a stimulus. Helper T cells, also known as CD4⁺ T cells, assist other types of white blood cells, playing roles such as assisting with the maturation of B cells into plasma and memory B cells, and activation of cytotoxic T cells and macrophages. Helper T cells are activated by the presentation of an antigen on the surface of an antigen-presenting cell (APC). Cytotoxic (killer) T cells, also known as CD8⁺ T cells, destroy virus-infected cells and tumor cells, and are associated with transplant rejection. Memory T cells recognize foreign invaders such as bacteria and viruses, and also tumor cells. After memory T cells encounter and respond to their cognate antigen, upon a second encounter, these cells are able to reproduce and mount a stronger and fast immune response. Regulatory (suppressor) T cells are critical for maintaining immunological tolerance; their major role is to downregulate T lymphocyte-mediated immunity near the end of an immune reaction and to inhibit autoreactive T lymphocytes that escaped the process of negative selection in the thymus. Natural killer T cells, which bridge the adaptive and innate immune systems, recognize an antigen presented by CD1d molecules and perform functions such as cytokine production and the release of cytolytic molecules.

As used herein, the terms “T lymphocyte receptor” and “T cell receptor (TCR)” refer to a molecule found on the surface of T cells that is responsible for recognizing fragments of antigens as peptides bound to major histocompatibility complex (MHC) molecules. Cross-linking of the TCRs by the peptide-MHC complex initiates the activation cascade of protein tyrosine kinases (PTKs), which in turn mediates the activation of a series of signaling molecules (28). Inositol phospholipid-specific phospholipase γ (PLCγ) is a key signaling molecule that is activated at the early stages of TCR signal transduction. Following TCR engagement, PLCγ is recruited to the TCR signaling complex, and is phosphorylated on tyrosine residues to become activated (29). Activated PLCγ hydrolyzes phosphatidylinositol 4,5-bisphosphate to generate diacylglycerol (DAG), inositol 1,4,5-trisphosphate (IP₃) (30) and cyclic adenosine diphosphate ribose (cADPR) (31). IP₃ and cADPR mediate calcium mobilization from the intracellular store via IP3 receptors (IP3Rs) and ryanodine receptors (RyRs), respectively. Stimulation of the activation cascade via TCR engagement with a TCR agonist stimulates transcription and expression of specific proteins (including RyRs) and T cell proliferation (e.g., expansion). Activated T cells can be re-stimulated with a TCR agonist to reactivate the activation cascade.

B lymphocytes secrete antibodies and function in the humoral immunity component of the adaptive immune system. In addition, B lymphocytes can function as antigen-presenting cells and secrete cytokines. B lymphocytes, which mature in the bone marrow, are distinguished from other types of lymphocytes by the presence of B cell receptors on the cell surface. Activation of B lymphocytes can either be T lymphocyte-dependent or T lymphocyte-independent. B lymphocytes are subdivided into several types, including plasmablast cells, plasma cells, lymphoplasmacytoid cells, memory B cells, follicular B cells, marginal zone B cells, B-1 cells, B-2 cells, and regulatory B (Breg) cells.

NK cells, which are part of the innate immune system, play major roles in defending against tumor cells and virally infected cells, differentiating these cells from normal cells by recognizing cell surface changes in major histocompatibility complex class I molecules. NK cells are activated by the interferon family of cytokines, and once activated release cytotoxic molecules that destroy the altered or infected cells.

The term “ryanodine receptor (RyR)” refers to a protein that releases calcium from the endoplasmic reticulum (ER) and sarcoplasmic reticulum (SR), has a high affinity for the plant alkaloid ryanodine, and in mammals is encoded by one of three genes: RYR1 (also known as MHS, MHS1, and CCO), RYR2, and RYR3. Ryanodine receptor isoform 1 (RyR1) is expressed primarily in skeletal muscle. Ryanodine receptor isoform 2 (RyR2) is expressed primarily in cardiac muscle. Ryanodine receptor isoform 3 (RyR3) is expressed more widely, especially in brain tissue. All three RyR isoforms are expressed in human lymphocytes (10). An example of a human RyR1 mRNA sequence is set forth under NCBI Reference Sequence No. NM_000540.2→NP_000531.2. An example of a human RyR2 mRNA sequence is set forth under NCBI Reference Sequence No. NM_001035.2→NP_001026.2. An example of a human RyR3 mRNA sequence is set forth under NCBI Reference Sequence No. NM_001036.4→NP_001027.3.

The term “inositol trisphosphate receptor (IP3R)” (also known as an “inositol 1,4,5-trisphosphate receptor”) refers to a membrane glycoprotein complex that acts as a calcium channel and is activated by inositol trisphosphate (IP3) (also known as inositol 1,4,5-trisphosphate). Type 1 and 2 IP3Rs are encoded by the ITPR1 and ITPR2 genes, respectively. Non-limiting examples of ITPR1 and ITPR2 human nucleotide sequences are set forth under NCBI Reference Sequence Nos. NM_002222.5→NP_002213.5 and NM_002223.3→NP_002214.2, respectively. IP3Rs are primarily located in the membrane of the endoplasmic reticulum (ER) of a large number of cell types, including lymphocytes, and facilitate the release of calcium from the ER.

The term “antagonist of calcium influx” means any agent that inhibits, reduces, or eliminates the movement of calcium ions into a cell (e.g., a lymphocyte such as a T lymphocyte). As a non-limiting example, an antagonist of calcium influx can be an extracellular solution or environment that makes calcium influx unfavorable (e.g., a low concentration of calcium ions such that inward movement of calcium ions through proteins (e.g. store-operated calcium channels or TRP channels) is not favorable). As another non-limiting example, an antagonist of calcium influx can be an agent, such as a calcium chelating agent, that binds calcium ions and inhibits or prevents calcium ions from entering a cell. As another non-limiting example, an antagonist of calcium influx can be any agent or compound that acts on a protein or other conduit of inward calcium ion movement into a cell (e.g., agents or compounds that inhibit calcium channels such as store-operated calcium channels or TRP channels). One of skill in the art will readily appreciate that different antagonists of calcium influx can be combined, as appropriate (e.g., a calcium chelating agent can be added to a low-calcium or calcium-free solution).

A “chelating agent” or “chelator” refers to an ion, molecule, or agent that forms a bond with a metal ion. Typically, chelation occurs by the formation of two or more coordinate bonds between the chelating agent and the metal ion. As a non-limiting example, a chelating agent can form bonds with a large number of metal ions (including both monovalent and divalent cations). As another non-limiting example, a chelating agent is much more specific for divalent cations than monovalent cations. As another non-limiting example, a chelating agent is much more specific for one divalent cation than another divalent cation (e.g., a chelating agent that is much more specific for Ca²⁺ than Mg²⁺). In the context of the present invention, the chelating agent is preferably a “calcium chelating agent” (i.e., a chelating agent that has high specificity for calcium over other metal ions).

The term “calcium-containing solution” refers to any solution that contains calcium. The calcium can be present in its ionized form, as a salt, or a combination thereof. Preferably, the concentration of calcium present in the solution is physiologically compatible with a cell (e.g., a lymphocyte such as a T lymphocyte) that is in contact with the solution. In some embodiments, the calcium-containing solution is isotonic. The calcium-containing solution can also contain, as non-limiting examples, other ions or salts thereof, buffers, nucleotides (e.g., adenosine triphosphate (ATP), sugars (e.g., glucose), proteins (e.g., serum), or other compounds.

The term “calcium-free solution” means a solution that is essentially free of calcium ions. In some embodiments, “essentially free of calcium” means that the concentration of calcium ions in the solution is less than about 0.1 mM (e.g., less than about 0.1, 0.09, 0.08, 0.07, 0.06, 0.05, 0.04, 0.03, 0.02, or 0.01 mM). In some embodiments, “essentially free of calcium” means that the concentration of calcium in the solution is about 0 mM. In some embodiments, the term “essentially free” refers to the concentration or number of free (e.g., unbound) calcium ions in the solution.

The term “sarco/endoplasmic reticulum Ca²⁺-ATPase (SERCA)” refers to an ATPase that transports calcium ions from the cytosol into the endoplasmic reticulum (ER) or sarcoplasmic reticulum (SR) of a cell and is encoded by a family of three genes in humans: SERCA1, SERCA2, and SERCA3. The three genes encode about 10 isoforms by alternative splicing. The SERCA1 isoform is specific for the SR in fast-twitch skeletal muscle. SERCA2 is found in the SR of slow-twitch skeletal muscle and in other tissues. The SERCA2a isoform is expressed in the SR of cardiac muscle, while the SERCA2b isoform is the major isoform expressed in smooth muscle and non-muscle tissues such as the epidermis. The three isoforms encoded by the SERCA3 gene vary in their C-termini and are often co-expressed with SERCA2b. SERCA2 and SERCA3 have been identified as being present in T lymphocytes.

The term “SERCA inhibitor” refers to any compound or agent that inhibits or eliminates SERCA function (e.g., the transport of calcium ions into the ER or SR from the cytosol). A SERCA inhibitor can be specific for a particular isoform of SERCA, or can inhibit many or all isoforms of SERCA. As a non-limiting example, a SERCA inhibitor inhibits or prevents calcium binding to SERCA and/or inhibits or prevents SERCA phosphorylation. As another non-limiting example, a SERCA inhibitor stabilizes SERCA in a particular configuration (e.g., the E1 or E2 configuration).

The term “calcium channel inhibitor” refers to any compound or agent that inhibits or eliminates calcium channel function. Preferably, the calcium channel inhibitor inhibits, reduces, or eliminates the influx of calcium ions into a cell (e.g., a lymphocyte such as a T lymphocyte) through a calcium channel. Calcium channel inhibitors can act on multiple types of calcium channels (e.g., store-operated calcium channels, TRP channels, voltage-gated calcium channels), or can be specific for a particular type of calcium channel.

The term “disorder related to abnormal intracellular calcium store discharge” refers to any disorder, disease, or pathological condition that is caused by, at least in part, abnormal discharge or release of calcium (i.e., greater than the amount of calcium discharge or release that occurs during normal physiological conditions) from intracellular calcium stores (e.g., endoplasmic reticulum, sarcoplasmic reticulum). In some embodiments, the disorder results, at least in part, from defective IP3 receptor function. In some embodiments, the disorder results, at least in part, from defective ryanodine receptor function. In some embodiments, a ryanodine receptor-related disorder results from one or more mutations in a gene encoding a ryanodine receptor (RyR) (e.g., RYR1, RYR2, RYR3) that lead to a RyR protein having aberrant functionality. In some embodiments, an IP3 receptor-related disorder results from one or more mutations in a gene encoding an IP3 receptor (IP3R) (e.g., IP3R1, IP3R2, IP3R3) that lead to an IP3R protein having aberrant functionality. In some embodiments, a RyR- or IP3R-related disorder results from a post-translational modification of an RyR or IP3R protein that lead to an RyR or IP3R protein having aberrant functionality. In many instances, defective or non-functional RyR and/or IP3R proteins lead to intracellular calcium signaling dysregulation, which in turn leads to pathological activation or inactivation of other cellular proteins or pathways. As non-limiting examples, defects in RyR and/or IP3R are associated with neurodegenerative diseases (e.g., Alzheimer's disease, Parkinson's disease, Huntington's disease), malignant hyperthermia, central core disease, multi-minicore disease, nemaline rod myopathy, T lymphocyte-mediated autoimmune disorders, polymorphic ventricular tachycardia, arrhythmogenic right ventricular dysplasia, heart failure, muscle fatigue, Duchenne muscular dystrophy, cancer, diabetes, and exposure to chemical pollutants. The term also includes disorders, diseases, and pathological conditions that are the result of RyR and/or IP3R dysfunction caused by exposure to various chemical pollutants.

Unless specified otherwise, the term “intracellular calcium” refers to cytosolic calcium ions, and does not include calcium ions located in the endoplasmic reticulum, sarcoplasmic reticulum, or mitochondria. In particular embodiments, the term also excludes bound calcium (e.g., calcium that is not in ionized form (e.g., bound to a protein or chelator)).

The term “store-operated calcium channel” (also known as a “calcium-release activated channel”) refers to a group of plasma membrane ion channels that are activated in response to the depletion of calcium from the endoplasmic reticulum. These channels are encoded by the genes ORAI1, ORAI2, and ORAI3, and respond to the interaction with STIM proteins, encoded by the genes STIM1, STIM2, and STIM3, which act as ER calcium sensors.

The term “transient receptor potential (TRP) channel” refers to a group of ion channels that share at least some structural similarity, are typically present on the plasma membrane of various animal cell types (including lymphocytes such as T lymphocytes) and are relatively non-selectively permeable to cations. The mammalian TRP family consists of 28 members divided into six subfamilies: 1 TRPA (ankyrin), 7 TRPC (canonical), 6 TRPV (vanilloid), 8 TRPM (melastatin), 3 TRPP (polycystin), and 2 TRPML (mucolipin). Most TRP channels contain 6 membrane-spanning helices and intracellular N- and C-termini. In mammals, TRP channels are activated or regulated by a wide variety of stimuli, including phosphorylation, G-protein receptor coupling, ligand-gating, and ubiquination.

The term “calcium-mobilizing agent” refers to any compound, composition, or agent that promotes or causes net movement of calcium ions directed from the intracellular store into the cytosol. In some embodiments, the calcium-mobilizing agent promotes the release or efflux of calcium ions from the intracellular calcium store (e.g., endoplasmic reticulum, sarcoplasmic reticulum, or endosomes). In some embodiments, the calcium-mobilizing agent inhibits or prevents the uptake of calcium ions from the cytosol into the intracellular calcium store (e.g., endoplasmic reticulum, sarcoplasmic reticulum, or endosomes).

### III. Methods of Determining Disorder Likelihood

In one aspect, the invention provides methods for determining whether a subject has an increased likelihood of having a disorder related to abnormal calcium discharge from the intracellular calcium store. In some embodiments, the method comprises:

(a) detecting an intracellular calcium concentration F₂ in a lymphocyte obtained from the subject while the lymphocyte is in contact with an antagonist of calcium influx;
(b)(1) detecting an intracellular calcium concentration F₁ in the lymphocyte while the lymphocyte is in contact with a calcium-containing solution, and/or
(b)(2) detecting an intracellular calcium concentration F₃ in the lymphocyte after the lymphocyte has been contacted with a calcium-mobilizing agent, or before and after the lymphocyte has been contacted with the calcium-mobilizing agent;
(c)(1) determining an intracellular calcium concentration difference Δ₁, wherein Δ₁ equals F₁ minus F₂, and/or
(c)(2) determining an intracellular calcium concentration difference Δ₂, wherein Δ₂ equals F₃ minus F₂; and
(d) determining whether the subject has an increased likelihood of having a disorder related to abnormal intracellular calcium store discharge based upon the value of Δ₁ and/or Δ₂.

In some embodiments, step (b)(2) is performed and F₃ is detected after the lymphocyte is in contact with the calcium-mobilizing agent. In some embodiments, step (b)(2) is performed and F₃ is detected before and after the lymphocyte has been contacted with the calcium-mobilizing agent. In some embodiments, step (b)(2) is performed and F₃ is detected while the lymphocyte is in contact with the calcium-mobilizing agent.

In some embodiments, only F₁ and F₂ are detected. In some embodiments, only F₂ and F₃ are detected. In some embodiments, F₁, F₂, and F₃ are detected. In some embodiments, only Δ₁ is used to make the determination of whether the subject has an increased likelihood of having a disorder related to abnormal intracellular calcium store discharge. In some embodiments, only Δ₂ is used to make the determination of whether the subject has an increased likelihood of having a disorder related to abnormal intracellular calcium store discharge. In some embodiments, both Δ₁ and Δ₂ are used to make the determination of whether the subject has an increased likelihood of having a disorder related to abnormal intracellular calcium store discharge.

In some embodiments, a different lymphocyte is used in step (a) (i.e., F₂ is detected while a different lymphocyte is in contact with an antagonist of calcium influx), step (b)(1) (i.e., F₁ is detected while a different lymphocyte is in contact with a calcium-containing solution), and/or step (b)(2) (i.e., F₃ is detected after a different lymphocyte has been contacted with a calcium-mobilizing agent or before and after a different lymphocyte has been contacted with a calcium-mobilizing agent). In some embodiments, the same lymphocyte is used in steps (a) and (b)(1) and a different lymphocyte is used in step (b)(2). In some embodiments, the same lymphocyte is used in steps (a) and (b)(2) and a different lymphocyte is used in step (b)(1). In some embodiments, only steps (a) and (b)(1) are performed and a different lymphocyte is used in each of steps (a) and (b)(1). In some embodiments, only steps (a) and (b)(2) are performed and a different lymphocyte is used in each of steps (a) and (b)(2). In some embodiments, steps (a), (b)(1), and (b)(2) are performed and a different lymphocyte is used in each of steps (a), (b)(1), and (b)(2).

In some embodiments, the same lymphocyte is used in all of steps (a), (b)(1), and/or (b)(2). In some embodiments, only steps (a) and (b)(1) are performed and the same lymphocyte is used in each of steps (a) and (b)(1) (i.e., F₂ is detected while the lymphocyte is in contact with an antagonist of calcium influx, and F₁ is detected while the same lymphocyte is in contact with a calcium-containing solution). In some embodiments, only steps (a) and (b)(2) are performed and the same lymphocyte is used in each of steps (a) and (b)(2) (i.e., F₂ is detected while the lymphocyte is in contact with an antagonist of calcium influx, and F₃ is detected after the same lymphocyte has been contacted with a calcium-mobilizing agent or before and after the same lymphocyte has been contacted with a calcium-mobilizing agent). In some embodiments, steps (a), (b)(1), and (b)(2) are performed, and the same lymphocyte is used in each of steps (a), (b)(1), and (b)(2) (i.e., F₂ is detected while the lymphocyte is in contact with an antagonist of calcium influx, F₁ is detected while the same lymphocyte is in contact with a calcium-containing solution, and F₃ is detected after the same lymphocyte has been contacted with a calcium-mobilizing agent or before and after the same lymphocyte has been contacted with a calcium-mobilizing agent).

In some embodiments, step (b)(1) is performed before step (a). In some instances, the lymphocyte is contacted with a calcium-containing solution and then the same lymphocyte is thereafter contacted with the antagonist of calcium influx. As a non-limiting example, the antagonist of calcium influx can be a solution that has a lower concentration than the calcium-containing solution (e.g., the antagonist of calcium influx is a low-calcium or calcium-free solution). In such instances, the lymphocyte can be transferred from one solution to another (e.g., the lymphocyte is transferred from a calcium-containing solution to a low-calcium or calcium-free solution), or the solution can be slowly or rapidly changed out (e.g., the lymphocyte is contacted with or contained in a bath solution, and the bath solution can be slowly or rapidly changed from a calcium-containing solution to a low-calcium or calcium-free solution).

In some embodiments, step (b)(2) is performed after step (a). In particular embodiments, step (b)(1) is performed before step (a), and then step (b)(2) is performed after step (a). As a non-limiting example, the lymphocyte is contacted with or contained in an antagonist of calcium influx (e.g., a low-calcium or calcium-free solution), and then the lymphocyte is contacted with a calcium-mobilizing agent (e.g., a SERCA inhibitor). In some instances, the lymphocyte is removed from contact with the antagonist of calcium influx before being contacted with the calcium-mobilizing agent. In particular embodiments, the lymphocyte is contacted with the calcium-mobilizing agent while the lymphocyte (e.g., the same lymphocyte) is in contact with the antagonist of calcium influx. The lymphocyte can be, as a non-limiting example, contacted with an antagonist of calcium influx (e.g., a low-calcium or calcium-free bath solution), and then a calcium-mobilizing agent (e.g., a SERCA inhibitor) can added to the bath solution such that the lymphocyte is brought into contact with or exposed to the calcium-mobilizing agent.

One of skill in the art will appreciate that Δ₁ can be determined at any time, as long as both F₁ and F₂ have been detected. As a non-limiting example, F₁ can be detected, followed by the detection of F₂, and then Δ₁ can be determined immediately or any other time after the detection of F₂. As another non-limiting example, F₂ can be detected, followed by the detection of F₁, and then Δ₁ can be determined immediately or any other time after the detection of F₁. Similarly, Δ₂ can be determined at any time, as long as both F₃ and F₂ have been detected. As a non-limiting example, F₃ can be detected, followed by the detection of F₂, and then Δ₂ can be determined immediately or any other time after the detection of F₂. As another non-limiting example, F₂ can be detected, followed by the detection of F₃, and then Δ₂ can be determined immediately or any other time after the detection of F₃.

As another non-limiting example, F₁, F₂, and F₃ can be detected, and then Δ₁ and Δ₂ can be determined thereafter. Furthermore, one of skill in the art will readily appreciate that a continuous intracellular concentration can be detected in a lymphocyte, and then the F₂ and F₁ and/or F₃ intracellular concentration values can be determined or assigned. Subsequently, the values of A1 and Δ₂ can be determined. As a non-limiting example, the intracellular concentration of a lymphocyte can be continuously detected while the lymphocyte is contacted with a calcium-containing solution followed by an antagonist of calcium influx, while the lymphocyte is contacted with an antagonist of calcium influx followed by a calcium-mobilizing agent, or while the lymphocyte is contacted with a calcium-containing solution, followed by an antagonist of calcium influx, followed by a calcium-mobilizing agent.

In some embodiments, F₂ is the lowest intracellular concentration detected before the lymphocyte is contacted with the calcium-mobilizing agent. As a non-limiting example, the intracellular calcium concentration can be detected or measured at any number of time points before the lymphocyte is contacted with the calcium-mobilizing agent, and then the lowest concentration that was detected or measured prior to the lymphocyte being contacted with the calcium-mobilizing agent can be taken to be or assigned as the F₂ intracellular calcium concentration.

The F₂ intracellular calcium concentration can be determined before or after application of the calcium-mobilizing agent and calcium mobilization from the intracellular calcium store is completed. In some embodiments, F₂ is lowest intracellular concentration detected before or after the lymphocyte is contacted (e.g., first contacted) with the calcium-mobilizing agent. The intracellular calcium concentration can be detected or measured at any number of time points before or after application of the calcium-mobilizing agent (e.g., SERCA inhibitor). In particular embodiments, detection is about 10 to 30 minutes (e.g., about 0, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30 minutes) before or after application of the calcium-mobilizing agent and/or after completion of the calcium discharge from the intracellular calcium store, and then the lowest concentration that was detected or measured before or after the lymphocyte was contacted with the calcium-mobilizing agent can be taken to be or assigned as the F₂ intracellular calcium concentration. One of skill in the art will readily be able to determine the appropriate time point or time interval for detecting F₂, depending on the particular calcium-mobilizing agent and other parameters.

The detection of intracellular concentration(s), the comparison of one or intracellular calcium concentrations detected at one or more time point(s), and/or the determination or assignment of an F₁, F₂ and/or F₃ intracellular calcium concentration value can be performed manually, can be automated, or a concentration thereof.

In some embodiments, F₃ is the highest intracellular concentration detected after the lymphocyte is contacted with the calcium-mobilizing agent. As a non-limiting example, the intracellular calcium concentration can be detected or measured at any number of time points while or after the lymphocyte is contacted with the calcium-mobilizing agent, and then the highest concentration that was detected or measured while or after the lymphocyte was contacted with the calcium-mobilizing agent can be taken to be or assigned as the F₃ intracellular calcium concentration. While F₃ can be detected at any suitable time after the lymphocyte has been contacted with the calcium-mobilizing agent, in some embodiments, F₃ is detected between about 0 and about 30 minutes (e.g., about 0, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30 minutes) after the lymphocyte is contacted (e.g., first contacted) with the calcium-mobilizing agent. In some embodiments, F₃ is detected more than 20 minutes after the lymphocyte is contacted with the calcium-mobilizing agent. In some embodiments, F₃ is detected between about 1 second and about 30 minutes (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 seconds or about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30 minutes) after the lymphocyte is contacted with the calcium-mobilizing agent. One of skill in the art will readily be able to determine the appropriate time point or time interval for detecting F₃, depending on the particular calcium-mobilizing agent and other parameters.

In some embodiments, F₃ is detected at two or more (e.g., about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 550, 600, 650, 700, 750, 800, 850, 900, 950, 1,000, 1,100, 1,200, 1,300, 1,400, 1,500, 1,600, 1,700, 1,800, 1,900, 2,000, 3,000, 3,500, 4,000, 4,500, 5,000, 5,500, 6,000, 6,500, 7,000, 7,500, 8,000, 8,500, 9,000, 9,500, 10,000, 20,000, 30,000, 40,000, 50,000, 60,000, 70,000, 80,000, 90,000, 100,000, or more) time points, wherein the earliest time point is t₁ and the latest time point is t₂.

Typically, t₂ will be a time point coincident with or after the time that the lymphocyte is contacted with the calcium-mobilizing agent. In some embodiments, t₂ is between about 0 and 30 minutes (e.g., about 0, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30 minutes) after the lymphocyte is contacted with the calcium-mobilizing agent. In some embodiments, t₂ is between about 5 and 10 minutes after the lymphocyte is contacted with the calcium-mobilizing agent. In some embodiments, t₂ is more than 30 minutes after the lymphocyte is contacted with the calcium-mobilizing agent.

In some embodiments, t₁ is a time point coincident with or after the time that the lymphocyte is contacted with the calcium-mobilizing agent. In some embodiments, t₁ is a time point before the lymphocyte is contacted with the calcium-mobilizing agent. In some embodiments, t₁ is between about 0 and 30 minutes (e.g., about 0, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30 minutes) before the lymphocyte is contacted with the calcium-mobilizing agent. In some embodiments, t₁ is more than 30 minutes before the lymphocyte is contacted with the calcium-mobilizing agent. In some embodiments, t₁ is between about 5 and 10 minutes before the lymphocyte is contacted with the calcium-mobilizing agent. One of skill in the art will recognize that the choice of t₁ and/or t₂ will be dictated, at least in part, by the choice of calcium-mobilizing agent, particularly when the intracellular calcium response to the calcium-mobilizing agent is transient (e.g., when a SERCA inhibitor is used as the calcium-mobilizing agent).

An intracellular calcium concentration (e.g., F₁, F₂, F₃) can be detected or measured as one or more absolute values, or alternatively can be integrated over a period of time (e.g., integrated over the period of time between t₁ and t₂).

In some embodiments, the lymphocyte is isolated from a sample (e.g., a sample that is obtained from the subject. The sample can be obtained by venipuncture, obtaining a volume of arterial blood, biopsy (e.g., needle or core biopsy), surgical resection, or any other suitable method. The sample can be a solid tissue sample, a fluid sample, or a combination thereof. Non-limiting examples of suitable samples include blood (e.g., whole blood), lymph tissue or fluid, tonsillar tissue, lung tissue, and mucosal tissue (e.g., intestinal mucosal surface tissue, oral mucosal tissue), or any combination thereof, so long as the sample contains a sufficient number of lymphocytes to enable the detection of the F₂ and F₁ and/or F₃ intracellular calcium concentrations.

In some embodiments, the methods of the present invention comprise detecting or measuring one or more intracellular calcium concentrations in a peripheral blood mononuclear cell (PBMC) such as a lymphocyte. In some embodiments, the lymphocyte is a T lymphocyte, a B lymphocyte, or a combination thereof. In preferred embodiments, the methods of the present comprise detecting or measuring one or more intracellular concentrations in a T lymphocyte, owing to the greater availability of T lymphocytes compared to B lymphocytes.

In some embodiments, the lymphocyte comprises a plurality of lymphocytes. In some embodiments, the plurality of lymphocytes is isolated from different samples that are obtained from the subject. In preferred embodiments, the plurality of lymphocytes is isolated from a single sample that is obtained from the subject. As a non-limiting example, when a different lymphocyte or plurality thereof is used in steps (a), (b)(1), and/or (b)(2), the different lymphocytes or pluralities thereof are all isolated from the same sample that is obtained from the subject.

Suitable calcium-containing solutions will readily be known to one of skill in the art. The calcium-containing solution can be a standard or commercially available solution, non-limiting examples being a solution (e.g. a physiological saline solution) such as Ringer's solution, Tyrode's solution and Hank's solution. The calcium-containing solution can also be a modified version of a standard or commercially available solution. Alternatively, any number of custom-made solutions can be used as the calcium-containing solution.

In some embodiments, the concentration of calcium in the calcium-containing solution is between about 0.1 mM and about 100 mM (e.g, about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mM). In some embodiments, the concentration of calcium in the calcium-containing solution is between about 0.1 mM and 50 mM. In some embodiments, the concentration of calcium in the calcium-containing solution is between about 0.1 mM and 10 mM. In some embodiments, the concentration of calcium in the calcium-containing solution is between about 2 mM and about 10 mM. In particular embodiments, the concentration of calcium in the calcium-containing solution is 2 mM. One of ordinary skill in the art will recognize that in embodiments where the calcium-containing solution contains a buffer (e.g., a calcium buffer) or a chelator (e.g., a calcium chelator), that the buffer and/or the chelator may affect the calcium concentration (e.g., effective or available calcium concentration) in the calcium-containing solution, and will be able to choose the appropriate buffer and/or chelator accordingly (e.g., in order to achieve the desired calcium concentration).

Non-limiting examples of antagonists of calcium influx include low-calcium solutions, calcium-free solutions (e.g., calcium-free physiological saline solutions), calcium chelating agents, calcium channel blockers, and any combination thereof. Examples of calcium chelating agents include, but are not limited to, ethylene glycol-bis(β-aminoethyl ether)-N,N,N′,N′-tetraacetic acid (EGTA), ethylenediaminetetraacetic acid (EDTA), 1,2-bis(o-aminophenoxy)ethane-N,N,N′,N′-tetraacetic acid (BAPTA), N-Carboxymethyl-N′-(2-hydroxyethyl)-N,N′-ethylenediglycine (HEDTA, also known as HEEDTA), pentetic acid, diethylenetriaminepentaacetic acid (DTPA), and any combination thereof.

Calcium channel blockers suitable for the methods of the present invention can inhibit or block the function of, for example, store-operated calcium channels, transient receptor potential (TRP) channels, or a combination thereof. Non-limiting examples of suitable store-operated calcium channel blockers include La³⁺, Gd³+, 2,6-difluoro-N-(1-(2-phenoxybenzyl)-1H-pyrazol-3-yl)benzamide (GSK-5503A), an imidazole, 2-aminoethoxydiphenylborate (2-APB), capsaicin, 5-nitro-2-(3-phenylpropylamino)-benzoic acid (NPPB), a bistrifluoromethyl-pyrazole derivative, diethylstilbestrol (DES), bromenol lactone (BEL), a bile acid, (1-(5-chloronaphthalene-1-sulfonyl)homopiperazine (ML-9), 4-(N-[[(6-hydroxy-1-naphthalenyl)amino]thioxomethyl]-2-furancarboxamide (CPD5J), 1-(5-chloronaphthalenesulfonyl)homopiperazine hydrochloride, (N-propylargylnitrendipine (MRS 1845), (1-[2-(4-Methoxyphenyl)-2-[3-(4-methoxyphenyl)propoxy]ethyl-1H-imidazole hydrochloride (SKF 96365 HCl), N-[4-[3,5-bis(trifluoromethyl)-1H-pyrazol-1-yl]phenyl]-4-methyl-1,2,3-thiadiazole-5-carboxamide (YM 58483), 2,6-difluoro-N-(1-(4-hydroxy-2-(trifluoromethyl)benzyl)-1H-pyrazol-3-yl)benzamide (GSK-7975A), N-[4-[3,5-bis(trifluoromethyl)-1H-pyrazol-1-yl]phenyl]-3-fluoro-4-pyridinecarboxamide (Pyr6), and combinations thereof.

Non-limiting examples of suitable TRP channel blockers include La³⁺, Gd³+, 2-[[3-(trifluoromethyl)phenyl]amino]benzoic acid (flufenamic acid), 1-[(4-chlorophenyl)methyl]-2-(1-pyrrolidinylmethyl)-1H-benzimidazole hydrochloride (clemizole hydrochloride), N-butyl-1H-benzimidazol-2-amine hydrochloride (M 084 hydrochloride), 3-([1,4′-bipiperidin]-1′-ylmethyl)-7-bromo-N-(1-phenylcyclopropyl)-2-[3-(trifluoromethyl)phenyl]-4-quinolinecarboxamide (GSK 2193874), 1,2,3,6-tetrahydro-1,3-dimethyl-N-[4-(1-methylethyl)phenyl]-2,6-dioxo-7H-purine-7-acetamide (HC-030031), N-(2-aminoethyl)-N-(4-(benzyloxy)-3-methoxybenzyl)thiophene-2-carboxamide hydrochloride (M8-B), a mefenamic acid analog, 2-(3-methylphenyl)aminobenzoic acid (3-MFA), 4-methyl-2-(1-piperidinyl)-quinoline (ML204), 3-[[[(1R)-1-(4-fluorophenyl)ethyl](3-quinolinylcarbonyl)amino]methyl]-benzoic acid (PF-05105679), N-[4-[3,5-bis(trifluoromethyl)-1H-pyrazol-1-yl]phenyl]-4-methyl-benzenesulfonamide (Pyr10), N-(4-(4-isopropylpiperazin-1-ylsulfonyl)phenyl)-2-nitro-4-(trifluoromethyl)benzamide (RN-9893), 4-(N-(3-chloro-5-(trifluoromethyl)pyridin-2-yl)-N-(4-(trifluoromethoxy)benzyl)sulfamoyl)benzoic acid (RQ-00203078), GsMTx4, 1-[2-(4-methoxyphenyl)-2-[3-(4-methoxyphenyl)propoxy]ethyl-1H-imidazole hydrochloride (SKF 96365 hydrochloride), (1-[4-[(2,3,3-trichloro-1-oxo-2-propen-1-yl)amino]phenyl]-5-(trifluoromethyl)-1H-pyrazole-4-carboxylic acid) (Pyr3), 4-methyl-2-(1-piperidinyl)quinolone (ML 204), and combinations thereof.

Non-limiting examples of calcium-mobilizing agents that are suitable for the methods of the present invention include sarco/endoplasmic reticulum Ca²⁺-ATPase (SERCA) inhibitors, calcium ionophores, membrane receptor agonists, intracellular receptor agonists (e.g., T lymphocyte receptor agonists), intracellular calcium chelators, cell-permeable lipophilic esters thereof, caged analogs thereof, and combinations thereof. SERCA inhibitors that are suitable for the methods of the present invention include, but are not limited to, thapsigargin, cyclopiazonic acid (CPA), 2,5-di(t-butyl)-1,4-hydroquinone (BHQ), nonylphenol, bisphenol, bisphenol A, tetrabromobisphenol A (TBBPA), 4-chloro-m-cresol, orthovanadate, a flavonoid, 2-aminoethoxydiphenyl borate (2APB), curcumin, paxilline, alisol B, mastoparan, peptide M391, ivermectin, cyclosporin A, rapamycin, chlorpromazine, calmidazolium, fluphenazine, diethylstilbestrol (DES), 1,3-dibromo-2,4,6-tris(methylisothiouronium) benzene, stable analog of HA 14-1 (sHA 14-1), cell-permeable lipophilic esters thereof, caged analogs thereof, and combinations thereof. In particular embodiments, the SERCA inhibitor is thapsigargin or CPA. Suitable flavonoids include, but are not limited to, 3,6-dihydroxyflavone, quercitin, galangin, cell-permeable lipophilic esters thereof, caged analogs thereof, and combinations thereof.

T lymphocyte receptor agonists (also referred to as “T cell receptor agonists”) are molecules that bind to T lymphocyte receptors (also referred to as “T cell receptors”) and/or other proteins that are functionally associated with T lymphocyte receptors (e.g., accessory proteins such as CD3 or CD28), thus initiating the activation cascade. Suitable T lymphocyte receptor agonists include, but are not limited to, T lymphocyte receptor specific antigens, super antigens, phytohemagglutinin (PHA), concanavalin A (Con A), soluble or immobilized antibodies against cluster of differentiation 3 (CD3) or cluster of differentiation 28 (CD28), lipopolysaccharide (LPS), pokeweed mitogen (PWM), bacterial superantigen (Sag) (e.g., staphylococcal enterotoxins (SE), streptococcal superantigen (SSA), staphylococcal enterotoxin-like toxin (SET), mouse mammary tumor virus (MMTV), SAg Epstein-Barr virus (EBV) SAg), and combinations thereof. In some embodiments, the T lymphocyte receptor agonist is presented to a lymphocyte in a soluble form or in a surface immobilized form, or a combination thereof.

In some embodiments, the intracellular receptor agonist is an inositol 1,4,5-trisphosphate (Ins (1,4,5)P₃ or IP₃) receptor agonist such as Ins (1,4,5)P₃ and related compounds (e.g., Ins(2,4,5) P₃, Ins(4,5)P₂, Ins(1,4,5)PS₃), adenophostins, adenosine triphosphate (ATP) cell permeable lipophilic esters thereof, caged analogs thereof, and combinations thereof. In some embodiments, the intracellular receptor agonist is an RyR receptor agonist, non-limiting examples of which include ryanodine, caffeine, heparin, 4-chloro-m-cresol, cyclic adenosine diphosphate (ADP) ribose, nicotinic acid adenine dinucleotide phosphate (NAADP), ATP, A2383, calmodulin, a cell permeable lipophilic ester thereof, a caged analog thereof, and a combination of thereof.

In some embodiments, the calcium-mobilizing agent is present (e.g., when contacted with the lymphocyte) at a concentration between about 0.001 μM and about 100 μM (e.g., about 0.001, 0.002, 0.003, 0.004, 0.005, 0.006, 0.007, 0.008, 0.009, 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM). In some embodiments, the calcium-mobilizing agent is present at a concentration between about 0.1 mM and about 10 mM (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mM). In some embodiments, the calcium-mobilizing agent is present at a concentration of more than about 10 mM. In particular embodiments, the calcium-mobilizing agent is present at a concentration of about 30 μM to about 50 μM. One of skill in the art will appreciate that the particular concentration or amount of calcium-mobilizing agent to be used will depend on the particular calcium-mobilizing agent and other parameters, and will be able to select the necessary concentration or amount accordingly.

Suitable calcium ionophores for use in the methods of the present invention include, but are not limited to, ionomycin, A23187 (calcimycin), 4-bromo A23187, and combinations thereof. Suitable intracellular calcium chelators include, but are not limited to, N,N,N′,N′-tetrakis(2-pyridylmethyl)ethane-1,2-diamine (TPEN), EGTA, EDTA, HEDTA, HEEDTA, pentetic acid, BAPTA, cell-permeable lipophilic esters (e.g., acetoxymethyl esters (AMs)) thereof, caged analogs thereof, and combinations thereof. A non-limiting example of a suitable intracellular receptor agonist is inositol trisphosphate (IP3).

In some embodiments, the intracellular calcium concentration in the lymphocyte (e.g., the F₁, F₂, and/or F₃ intracellular calcium concentration) is detected by observing or recording a signal. In particular embodiments, the signal is displayed on a device (e.g., computer monitor or other electronic display) and/or recorded on an analog and/or digital recording medium. Other methods or devices for detecting a signal (e.g., optical signal, such as a fluorescent signal) include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, and signal amplification using photomultiplier tubes.

In some embodiments, the signal is proportional (e.g., directly proportional) to the intracellular calcium concentration. In other embodiments, the signal is inversely proportional to the intracellular calcium concentration. In some embodiments, the signal is an optical (e.g., fluorescent) signal. In particular embodiments, detecting a fluorescent signal comprises detecting a change in fluorescence (e.g., a change in fluorescence intensity, fluorescence excitation or emission wavelength distribution, fluorescent lifetime, and/or fluorescence polarization).

In some embodiments, the signal is generated using a fluorescent calcium indicator or sensor (e.g., a dye). In some embodiments, the lymphocyte is loaded with the fluorescent calcium indicator before the lymphocyte is contacted with the calcium-containing solution. In some embodiments, the lymphocyte is loaded with the fluorescent calcium indicator or sensor after the lymphocyte is contacted with the calcium-containing solution. In some embodiments, the lymphocyte is loaded with the fluorescent calcium indicator while the lymphocyte is contacted with the antagonist of calcium influx (e.g., calcium-free solution). In particular embodiments, the fluorescent calcium indicator or sensor signal is directly proportional to the intracellular calcium concentration. Non-limiting examples of suitable fluorescent calcium indicators or sensors include bis-fura-2, BTC, calcium green-1, calcium green-2, calcium green-5n, calcium orange, calcium crimson, fluo-3, fluo-4, fluo-5F, fluo-4FF, fluo-5N, fura-2, fura-4f, fura-6F, fura-FF, fura red, indo-1, mag-fluo-4, mag-fura-2, mag-indo-1, magnesium green, Oregon green 488 BAPTA-1, Oregon green 488 BAPTA-2, Oregon green 488 BAPTA-6F, Oregon green 488 BAPTA-5N, quin-2, rhod-2, rhod-3, rhod-FF, rhod-5N, X-rhod-1, X-rhod-5F, a genetically-encoded sensor, analogs thereof, acetoxymethyl esters thereof, acetate esters thereof, water soluble salts thereof, dextran variants thereof, cadaverine variants thereof, thiol-reactive iodoacetamides thereof, and combinations thereof.

In some embodiments, the fluorescent calcium indicator or sensor is a non-ratiometric dye. In such instances, the intracellular calcium concentration (e.g., free intracellular calcium concentration) is determined by a relative increase in fluorescence intensity. Non-ratiometric dyes primarily work in the visible wavelength range, and the use of a single excitation wavelength allows for simpler instrumentation and/or simultaneous observation of other parameters. In some embodiments, the relationship between fluorescence and the absolute calcium concentration (e.g., absolute intracellular calcium concentration) is represented by the following equation:

[Ca²⁺]=K_{d}×(F−Fₘᵢₙ)/(Fₘₐₓ−F)

where K_{d} is the dissociation constant of the fluorescent indicator and Fₘᵢₙ and Fₘₐₓ are fluorescence values obtained at minimum and maximum calcium concentrations (e.g., 0 and saturating values). K_{d} is commonly determined for each experimental condition, as K_{d} can be modified by factors such as pH, protein concentration, ionic strength, and lipid interactions. Calcium chelators are often used to obtain accurate Fₘᵢₙ values.

Non-limiting examples of non-ratiometric calcium dyes include calcium green-1, calcium green-2, calcium green-5N, calcium orange, fluo-3, fluo-4, fluo-5F, fluo-4FF, fluo-5N, mag-fluo-4, magnesium green, Oregon green 488 BAPTA-1, Oregon green 488 BAPTA-2, Oregon green 488 BAPTA-6F, Oregon green 488 BAPTA-5N, quin-2, rhod-2, rhod-3, rhod-FF, rhod-5N, X-rhod-1, and X-rhod-5F. Exemplary emission wavelengths for non-ratiometric calcium dyes include 495, 520, 525, 530, 535, 575, 580, 600, and 615 nm.

In some embodiments, the fluorescent calcium indicator or sensor is a ratiometric dye. In such instances, the intracellular calcium concentration is measured as a ratio between two fluorescence intensity values that are measured at two different excitation or emission wavelengths. Ratiometric dyes allow for correction for unequal dye loading, bleaching, and focal plane shift. Non-limiting examples of ratiometric calcium dyes include bis-fura-2, BTC, fura-2, fura-4f, fura-6F, fura-FF, fura red, indo-1, mag-fura-2, and mag-indo-1. Exemplary pairs of excitation frequencies for ratiometric dyes include 340 and 380 nm, 400 and 480 nm, 420 and 480 nm, and 405 and 485 nm.

In some embodiments, the methods of the present invention further comprise contacting the lymphocyte with a lymphocyte activating agent. In some embodiments, the methods of the invention comprise detecting intracellular calcium concentration(s) (e.g., F₁, F₂, and/or F₃) in activated lymphocytes. In certain instances, for example when a limited number of activated lymphocytes are available in a sample (e.g., in a sample obtained from a subject), it may be desirable to employ one or more lymphocyte activating agents to make previously unactivated lymphocytes present in the sample available for use according to the methods of the present invention. For example, unactivated lymphocytes often produce a poor signal-to-noise ratio when calcium fluorescence is used to detect intracellular calcium concentration, but contacting unactivated lymphocytes with an activating agent can improve the signal-to-noise ratio such that the previously unactivated lymphocytes become useful for detecting intracellular calcium concentrations. Suitable lymphocyte activating agents include, but are not limited to, ionomycin, phorbol myristate acetate (PMA), phytohemagglutinin (PHA), lipopolysaccharide (LPS), concanavalin A (Con A), pokeweed mitogen (PWM), an anti-CD3 antibody, an anti-CD28 antibody, bacterial superantigen (SAg; e.g., staphylococcal enterotoxins (SE); streptococcal superantigen (SSA); staphylococcal enterotoxin-like toxin (SET); mouse mammary tumor virus (MMTV) SAg; Epstein-Barr virus (EBV) SAg), and a combination thereof. In some embodiments, the TCR agonist is presented to a lymphocyte in a soluble form, in the surface immobilized form, or a combination thereof.

The methods of the present invention can be used to determine whether a subject has an increased likelihood of having a disorder related to abnormal intracellular calcium discharge. Non-limiting examples of suitable diseases include Alzheimer's disease (AD), Parkinson's disease, Huntington's disease, malignant hyperthermia (MH), central core disease, multi-minicore disease (MmD), nemaline rod myopathy (NM), heart failure, muscle fatigue, Duchenne muscular dystrophy, cancer, diabetes, a T lymphocyte-mediated autoimmune disease, and exposure to a chemical pollutant.

Non-limiting examples of chemical pollutants for which exposure can be detected according to the methods of the present invention include polychlorinated biphenyl compound, triclosan, bisphenol A, and a combination thereof.

T lymphocyte-mediated autoimmune diseases for which the methods of the present invention can be used to determine an increased likelihood in a subject include, but are not limited to, autoimmune nervous system diseases (e.g., multiple sclerosis (MS), myasthenia gravis, autoimmune neuropathies such as Guillian-Barre syndrome), autoimmune ophthalmologic diseases (e.g., uveitis), autoimmune blood disorders (e.g., autoimmune hemolytic anemia, pernicious anemia, autoimmune thrombocytopenia), autoimmune vascular diseases (e.g., temporal arteritis, anti-phospholipid syndrome, autoimmune vasculitis, Bechet's disease, atherosclerosis), autoimmune skin diseases (e.g., psoriasis, dermatitis herpetiformis, pemphigus vulgaris, vitiligo, mycosis fungoides, allergic contact dermatitis, atopic dermatitis, lichen planus, pityriasis lichenoides at varioliforms acute (PLEVA)), autoimmune gastrointestinal diseases (e.g., Crohn's disease, ulcerative colitis, primary biliary cirrhosis, autoimmune hepatitis), and autoimmune endocrine disorders (e.g., type I diabetes mellitus, Addison's disease, Grave's disease, Hashimoto's thyroiditis). In some embodiments, the method further comprises providing a prognosis or diagnosis (e.g., as to whether the subject has an increased likelihood of having a disorder related to abnormal intracellular calcium discharge) to the subject.

In some embodiments, the determination of whether the subject has an increased likelihood of having a disorder related to abnormal calcium discharge from the intracellular calcium store is based upon the value of Δ₁. In some embodiments, the determination of whether the subject has an increased likelihood of having a disorder related to abnormal calcium store discharge is based upon the value of Δ₂. In some embodiments, the determination of whether the subject has an increased likelihood of having a disorder related to abnormal calcium store discharge is based upon a difference between Δ₁ and Δ₂ (e.g., Δ₁-Δ₂ or Δ₂-A1). In some embodiments, the determination of whether the subject has an increased likelihood of having a disorder related to abnormal calcium store discharge is based upon a ratio of Δ₁ to Δ₂ (i.e., Δ₁/Δ₂). In some embodiments, the value of Δ₁, Δ₂, the difference between Δ₁ and Δ₂, or the ratio of Δ₁ to Δ₂ is input into a regression model (e.g., logistic regression model) to make the determination of whether a subject has an increased likelihood of having a disorder related to abnormal calcium store discharge. In some embodiments, the value of Δ₁, Δ₂, the difference between Δ₁ and Δ₂, or the ratio of Δ₁ to Δ₂ is compared to a reference value. In particular embodiments, the reference value is determined from a control subject or a control population (e.g., a subject or population of subjects who do not have a disorder related to abnormal intracellular calcium store discharge).

In some embodiments, a subject is determined to have an increased likelihood of having a disorder related to abnormal calcium store discharge when the value of Δ₁ is higher than a reference value. In particular embodiments, the subject is determined to have an increased likelihood of having a disorder related to abnormal calcium store discharge when the value of Δ₁ is at least about 1.1×, 1.2×, 1.3×, 1.4×, 1.5×, 1.6×, 1.7×, 1.8×, 1.9×, 2×, 2.5×, 3×, 3.5×, 4×, 4.5×, 5×, 5.5×, 6×, 6.5×, 7×, 7.5×, 8×, 8.5×, 9×, 9.5×, 10×, 11×, 12×, 13×, 14×, 15×, 16×, 17×, 18×, 19×, or 20× the reference value. In other embodiments, the subject is determined to have an increased likelihood of having a disorder related to abnormal calcium store discharge when the value of Δ₁ is at least about 1, 2, 3, or more standard deviations greater than the reference value.

In some embodiments, a subject is determined to have an increased likelihood of having a disorder related to abnormal calcium store discharge when the value of Δ₂ is lower than a reference value. In particular embodiments, the subject is determined to have an increased likelihood of having a disorder related to abnormal calcium store discharge when the value of Δ₂ is at less than about 0.95×, 0.9×, 0.85×, 0.8×, 0.75×, 0.7×, 0.65×, 0.6×, 0.55×, 0.5×, 0.45×, 0.4×, 0.35×, 0.3×, 0.25×, 0.2×, 0.15×, 0.1×, 0.09×, 0.08×, 0.07×, 0.06×, 0.05×, 0.04×, 0.03×, 0.02×, or 0.01× the reference value. In other embodiments, the subject is determined to have an increased likelihood of having a disorder related to abnormal calcium store discharge when the value of Δ₂ is at least about 1, 2, 3, or more standard deviations lower than the reference value.

In some embodiments, a subject is determined to have an increased likelihood of having a disorder related to abnormal calcium store discharge when the value of Δ₁-Δ₂ is higher than a reference value. In some embodiments, a subject is determined to have an increased likelihood of having a disorder related to abnormal calcium store discharge when the value of Δ₂-Δ₁ is lower than a reference value. In some embodiments, a subject is determined to have an increased likelihood of having a disorder related to abnormal calcium store discharge when the absolute value of Δ₁-Δ₂ or Δ₂-Δ₁ is higher than a reference value. In particular embodiments, the subject is determined to have an increased likelihood of having a disorder related to abnormal calcium store discharge when the absolute value of Δ₁-Δ₂ is at least about 1.1×, 1.2×, 1.3×, 1.4×, 1.5×, 1.6×, 1.7×, 1.8×, 1.9×, 2×, 2.5×, 3×, 3.5×, 4×, 4.5×, 5×, 5.5×, 6×, 6.5×, 7×, 7.5×, 8×, 8.5×, 9×, 9.5×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, or 40× the reference value. In other embodiments, the subject is determined to have an increased likelihood of having a disorder related to abnormal calcium store discharge when the absolute value of Δ₁-Δ₂ is at least about 1, 2, 3, or more standard deviations greater than the reference value.

In some embodiments, a subject is determined to have an increased likelihood of having a disorder related to abnormal calcium store discharge when the value of Δ₁/Δ₂ is higher than a reference value. In particular embodiments, the subject is determined to have an increased likelihood of having a disorder related to abnormal calcium store discharge when the value of Δ₁/Δ₂ is at least about 2×, 2.5×, 3×, 3.5×, 4×, 4.5×, 5×, 5.5×, 6×, 6.5×, 7×, 7.5×, 8×, 8.5×, 9×, 9.5×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, or 40× the reference value. In other embodiments, the subject is determined to have an increased likelihood of having a disorder related to abnormal calcium store discharge when the value of Δ₁/Δ₂ is at least about 1, 2, 3, or more standard deviations greater than the reference value.

### IV. Kits

The present invention also provides kits to facilitate executing the methods of the present invention. In some embodiments, the kits comprise materials, reagents and/or compositions for the preparation of calcium-containing solutions, antagonists of calcium influx, calcium-mobilizing agents, or solutions thereof. As used herein, the term “kit” includes a combination of articles that facilitates a process, assay, analysis, or manipulation.

Kits can contain chemical reagents as well as other components. In addition, the kits of the present invention can include, without limitation, instructions to the kit user, apparatus and reagents for sample collection and/or purification, apparatus and reagents for loading fluorescent dyes into cells, reagents and apparatus for detecting intracellular concentrations, sample tubes, holders, trays, racks, dishes, plates, solutions, buffers or other chemical reagents, suitable samples to be used for standardization, normalization, and/or control samples. Kits of the present invention can also be packaged for convenient storage and safe shipping, for example, in a box having a lid.

### V. Examples

The present invention will be described in greater detail by way of specific examples. The following examples are offered for illustrative purposes only, and are not intended to limit the invention in any manner.

### Example 1. Detecting Defective Intracellular Calcium Storage in T Lymphocytes

This example demonstrates the detection of defective intracellular calcium discharge from the intracellular calcium store in two different transgenic mouse lines. RyR163C mice have a mutation in the ryanodine receptor type 1, which increases calcium discharge from the intracellular store via RyRs. The RyR163C mice develop malignant hyperthermia (MH) in response to exposure to the volatile anesthetic halothane or elevated ambient temperatures. The 5×FAD transgenic mice overexpress mutant human APP(695) with the Swedish (K670N, M671L), Florida (I716V), and London (V717I) Familial Alzheimer's Disease (FAD) mutations along with human PS1 harboring two FAD mutations, M146L and L286V. The 5×FAD transgenic mice recapitulate major features of Alzheimer's disease pathology.

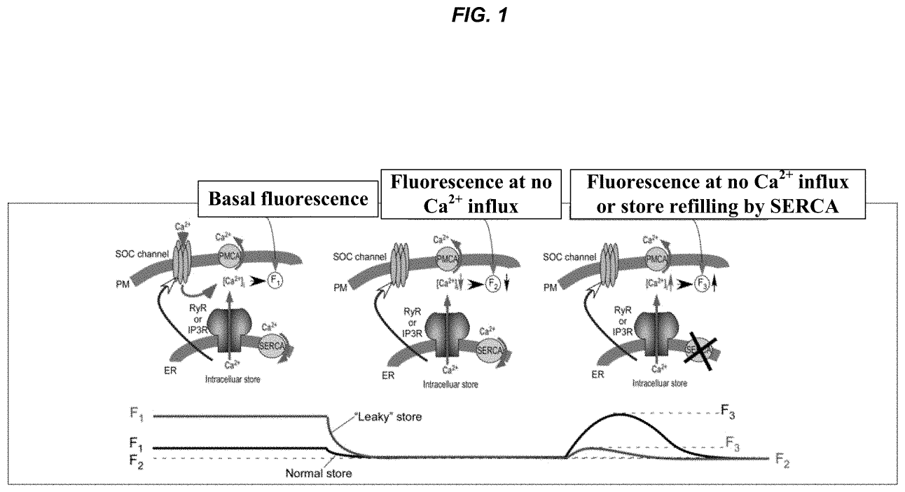

A number of human diseases, including Alzheimer's disease and malignant hyperthermia, are associated with mutations in proteins that regulate calcium discharge from the intracellular calcium store. At resting conditions, in normal T lymphocytes that are isolated from healthy subjects, the calcium discharge (or “leak”) from the intracellular calcium store (i.e., mostly from the endoplasmic reticulum (ER)) via inositol trisphosphate receptors (IP3Rs) and ryanodine receptors (RyRs) and calcium entry via plasma membrane store-operated calcium (SOC) channels is usually very small (14-17). However, the enhanced conductance or expression level of IP3Rs and/or RyRs, which has been observed in the presence of autosomal dominant Alzheimer's disease (ADAD) and malignant hyperthermia (MH) mutations, increases discharge from the calcium store, causing store depletion and activation of calcium entry via SOC channels (FIG. 1). Calcium influx via SOC channels elevates the intracellular calcium concentration to a new higher basal level such that the rates of calcium influx and extrusion by the plasma membrane calcium ATPase (PMCA) will match each other. Following the inhibition of SOC entry by the removal of extracellular calcium, the compensatory action of PMCA manifests itself as a decrease in basal intracellular calcium concentration (FIG. 1, lower traces). In the absence of calcium influx, enhanced discharge from the calcium store causes store depletion, which can be detected by probing the store content with SERCA blockers. Changes in the intracellular calcium concentration produce corresponding changes in fluorescence intensity (F) of a calcium indicator.

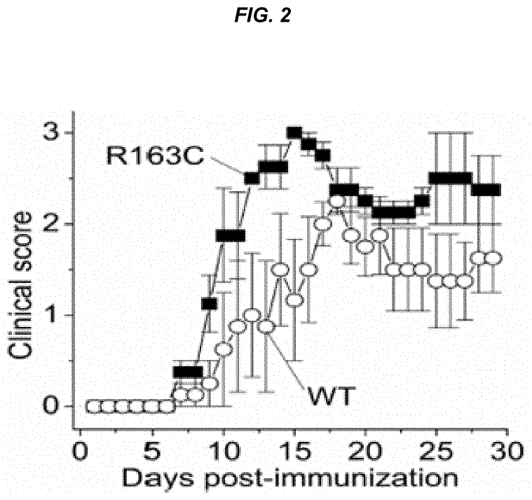

In this example, two different transgenic mouse lines (i.e., the 5×FAD and RyR163C transgenic mouse lines) were used. The 5×FAD and RyR163C mouse lines recapitulate many of the phenotypes of Alzheimer's disease (AD) and malignant hyperthermia (MH), respectively. FIG. 2 shows that the R163C RyR1 gain-of-function mutation increases splenocyte calcium signaling and affects T lymphocyte activation. In addition, the RyR1 gain-of-function mutation promotes a T lymphocyte-mediated autoimmune response.

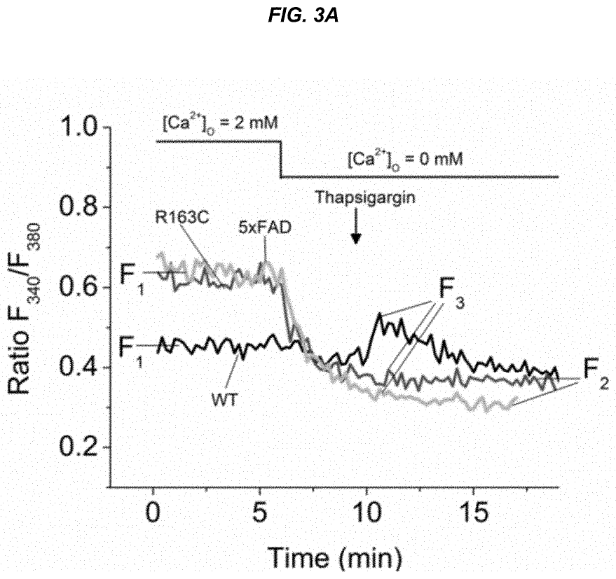

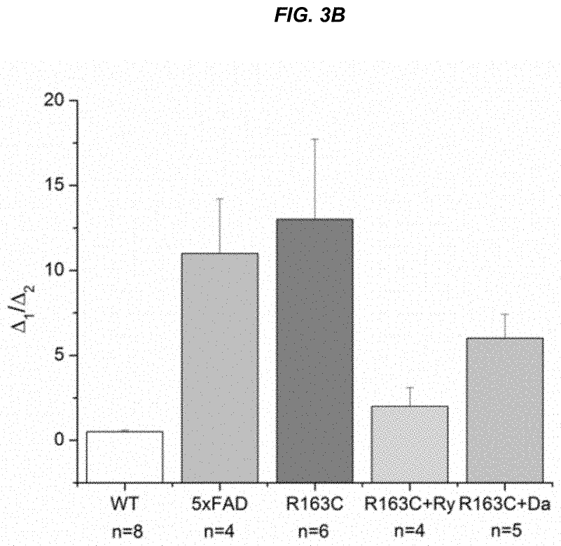

As shown in FIG. 3A, T lymphocytes that were isolated from the spleens and lymph nodes of 5×FAD and RyR163C mice displayed elevated basal intracellular calcium levels (determined using the fluorescent calcium indicator fura-2) compared to wild-type (WT) mice. Upon removal of extracellular calcium (i.e., decreasing the calcium concentration of the bath solution from about 2 mM to about 0 mM), the decrease in the intracellular calcium concentration was significantly larger in T lymphocytes isolated from 5×FAD and RyR163C mice than in T lymphocytes isolated from control WT mice (FIG. 3A; Δ₁=F₁−F₂). Upon application of the SERCA blocker thapsigargin, larger intracellular calcium transients were evoked in control WT cells compared to the barely detectable intracellular calcium transients that were observed in T lymphocytes isolated from the 5×FAD and RyR163C mice (FIG. 3B; Δ₂=F₃−F₂). RyR blockade with the RyR blocker ryanodine (800 μM) or dantrolene (50 μM) reduced the Δ₁/Δ₂ ratio by reducing the basal intracellular calcium levels and increasing the responses to thapsigargin in T lymphocytes isolated from RyR163C mice (FIG. 3B), indicating that calcium discharge is primarily mediated via RyRs. This experiment demonstrated a greater than 20-fold increase in the Δ₁/Δ₂ ratio in T lymphocytes from 5×FAD and RyR163C mice with “leaky” intracellular calcium stores compared to T lymphocytes from WT mice (FIG. 3B).

The experiments presented in this example show that the methods of the present invention enable strong discrimination between normal and “leaky” T lymphocyte calcium stores. The calcium responses recorded from T lymphocytes isolated from WT mice closely resembled those previously observed in PBMC-derived T lymphocytes from healthy humans and Jurkat T lymphocytes (a human leukemia T lymphocyte line), indicating that 1) the healthy murine T lymphocyte calcium handling system is similar to that of healthy humans, and 2) Jurkat T lymphocytes can be used for calibration of instrumentation.

### Example 2. Defective T Lymphocyte Intracellular Calcium Storage in Alzheimer's Disease and Malignant Hyperthermia

This example describes how methods of the present invention can be used to determine that a subject has an increased likelihood of developing a ryanodine receptor-related disorder.

### Alzheimer's Disease

Using methods of the present invention, the discharge of intracellular calcium stores in T lymphocytes can be measured and used to detect the presence of abnormal calcium regulation caused by mutations (e.g., causative mutations) associated with Alzheimer's disease (AD) in humans and other animals (e.g., mice). In particular, an increase in the Δ₁/Δ₂ ratio measured in T lymphocytes, detected by methods of the present invention, can be detected in advance of amyloid deposition and/or cognitive impairment in a subject (e.g., a humans or a rodent such as a mouse).

Previous studies performed on neurons and skin fibroblasts of pre-symptomatic humans and mice bearing autosomal dominant Alzheimer's disease (ADAD) mutations have shown that the appearance of clinical symptoms of AD is preceded by a dysregulation of intracellular calcium handling. In order to calibrate the relationship between an increase in the T lymphocyte Δ₁/Δ₂ ratio and the timing of the appearance of clinical symptoms, transgenic mice such as 5×FAD are used. Four-week old 5×FAD pre-symptomatic mice are obtained (e.g., from The Jackson Laboratory). One 5×FAD mouse is sacrificed every week, beginning from four weeks of age, through 24 weeks of age. Lymphoid organs (e.g., spleen and lymph nodes) and brains are collected for calcium imaging and immunostaining, respectively. T lymphocytes are isolated from the lymphoid organs (e.g., using the EasySep Mouse T Cell Isolation Kit (available from STEMCELL Technologies)). Calcium imaging is performed as shown in FIG. 1. The timing between a measurable increase in the Δ₁/Δ₂ ratio and amyloid deposition (assessed, for example, by immunostaining of the brain sections) and/or cognitive impairment (assessed, for example, by employing the Y maze test) is determined. Wild-type mice serve as a control.

### Malignant Hyperthermia

Data obtained from experiments performed with malignant hyperthermia (MH)-susceptible R163C mice, described above in Example 1, show that the Δ₁/Δ₂ ratio is higher in T lymphocytes isolated from MH-susceptible subjects than in T lymphocytes isolated from control subjects (FIG. 3). In order to calibrate the relationship between the Δ₁/Δ₂ ratio and MH susceptibility in humans, T lymphocytes isolated from blood samples obtained from healthy and MH-susceptible human subjects are used. Blood samples (e.g., 30 mL samples) are obtained from healthy subjects and subjects who are susceptible to MH, and CD3⁺ T lymphocytes are isolated using, as a non-limiting example, the EasySep Human T Cell Isolation Kit available from STEMCELL Technologies. The MH-susceptible subjects can be subjects who have had a clinical episode of MH or have a family history of MH. Measurement of intracellular calcium is measured in the isolated lymphocytes as shown in FIG. 1.

### VI. References

1. Vazquez Ruiz de Castroviejo, E., et al., [Analysis of the frequency of cardiac arrhythmias and conduction disturbances from a health-care perspective]. Rev Esp Cardiol, 2005. 58(6): p. 657-65.
2. Chakroborty, S. and G. E. Stutzmann, Calcium channelopathies and Alzheimer's disease: insight into therapeutic success and failures. Eur J Pharmacol, 2014. 739: p. 83-95.
3. Betzenhauser, M. J. and A. R. Marks, Ryanodine receptor channelopathies. Pflugers Arch, 2010. 460(2): p. 467-80.
4. Jurkat-Rott, K., H. Lerche, and F. Lehmann-Horn, Skeletal muscle channelopathies. J Neurol, 2002. 249(11): p. 1493-502.
5. Taratuto, A. L., Congenital myopathies and related disorders. Curr Opin Neurol, 2002. 15(5): p. 553-61.
6. Rosenberg, H., et al., Malignant hyperthermia. Orphanet J Rare Dis, 2007. 2: p. 21.
7. Kim, J. H., et al., Exome sequencing reveals novel rare variants in the ryanodine receptor and calcium channel genes in malignant hyperthermia families. Anesthesiology, 2013. 119(5): p. 1054-65.
8. Robinson, R. L., et al., Recent advances in the diagnosis of malignant hyperthermia susceptibility: how confident can we be of genetic testing? Eur J Hum Genet, 2003. 11(4): p. 342-8.
9. Rosenberg, H., J. F. Antognini, and S. Muldoon, Testing for malignant hyperthermia. Anesthesiology, 2002. 96(1): p. 232-7.
10. Bina, S., et al., Lymphocyte-based determination of susceptibility to malignant hyperthermia: a pilot study in swine. Anesthesiology, 2010. 113(4): p. 917-24.
11. Wong, P. W. and I. N. Pessah, Ortho-substituted polychlorinated biphenyls alter calcium regulation by a ryanodine receptor-mediated mechanism: structural specificity toward skeletal- and cardiac-type microsomal calcium release channels. Mol Pharmacol, 1996. 49(4): p. 740-51.
12. Ahn, K. C., et al., In vitro biologic activities of the antimicrobials triclocarban, its analogs, and triclosan in bioassay screens: receptor-based bioassay screens. Environ Health Perspect, 2008. 116(9): p. 1203-10.
13. Pessah, I. N., G. Cherednichenko, and P. J. Lein, Minding the calcium store: Ryanodine receptor activation as a convergent mechanism of PCB toxicity. Pharmacol Ther, 2010. 125(2): p. 260-85.
14. Thakur, P., S. Dadsetan, and A. F. Fomina, Bidirectional coupling between ryanodine receptors and Ca2+ release-activated Ca2+(CRAC) channel machinery sustains store-operated Ca2+ entry in human T lymphocytes. J Biol Chem, 2012. 287(44): p. 37233-44.
15. Van Petegem, F., Ryanodine receptors: allosteric ion channel giants. J Mol Biol, 2015. 427(1): p. 31-53.
16. Eltit, J. M., et al., Orthograde dihydropyridine receptor signal regulates ryanodine receptor passive leak. Proc Natl Acad Sci USA, 2011. 108(17): p. 7046-51.
17. Dadsetan, S., et al., Store-operated Ca2+ influx causes Ca2+ release from the intracellular Ca2+ channels that is required for T cell activation. J Biol Chem, 2008. 283(18): p. 12512-9.
18. Barrientos, G. C., et al., Gene dose influences cellular and calcium channel dysregulation in heterozygous and homozygous T48261-RYR1 malignant hyperthermia-susceptible muscle. J Biol Chem, 2012. 287(4): p. 2863-76.
19. Yuen, B., et al., Mice expressing T48261-RYR1 are viable but exhibit sex- and genotype-dependent susceptibility to malignant hyperthermia and muscle damage. Faseb J, 2012. 26(3): p. 1311-22.
20. Cherednichenko, G., et al., Enhanced Excitation-Coupled Calcium Entry (ECCE) in Myotubes Expressing Malignant Hyperthermia Mutation R163C is Attenuated by Dantrolene. Mol Pharmacol, 2008.
21. Thakur, P. and A. F. Fomina, Whole-Cell Recording of Calcium Release-Activated Calcium (CRAC) Currents in Human T Lymphocytes. J Vis Exp, 2010. 46.
22. MacFarlane, A. W. t., J. F. Oesterling, and K. S. Campbell, Measuring intracellular calcium signaling in murine NK cells by flow cytometry. Methods Mol Biol, 2010. 612: p. 149-57.
23. Briggs, C. A., S. Chakroborty, and G. E. Stutzmann, Emerging pathways driving early synaptic pathology in Alzheimer's disease. Biochem Biophys Res Commun, 2017. 483(4): p. 988-997.
24. Wehrens, X. H., et al., Protection from cardiac arrhythmia through ryanodine receptor-stabilizing protein calstabin2. Science, 2004. 304(5668): p. 292-6.
25. Bellinger, A. M., et al., Hypernitrosylated ryanodine receptor calcium release channels are leaky in dystrophic muscle. Nat Med, 2009. 15(3): p. 325-30.
26. Waning, D. L., et al., Excess TGF-beta mediates muscle weakness associated with bone metastases in mice. Nat Med, 2015. 21(11): p. 1262-1271.
27. Santulli, G., et al., Calcium release channel RyR2 regulates insulin release and glucose homeostasis. J Clin Invest, 2015. 125(11): p. 4316.
28. Samelson, L. E., Signal transduction mediated by the T cell antigen receptor: the role of adapter proteins. Annu Rev Immunol, 2002. 20: p. 371-94.
29. Nishibe, S., et al., Increase of the catalytic activity of phospholipase C-gamma 1 by tyrosine phosphorylation. Science, 1990. 250(4985): p. 1253-6.
30. Rhee, S. G., Regulation of phosphoinositide-specific phospholipase C. Annu Rev Biochem, 2001. 70: p. 281-312.
31. Guse, A. H., et al., Regulation of calcium signalling in T lymphocytes by the second messenger cyclic ADP-ribose. Nature, 1999. 398(6722): p. 70-3.

### VII. Exemplary Embodiments

Exemplary embodiments provided in accordance with the presently disclosed subject matter include, but are not limited to, the claims and the following embodiments:

1. A method for determining whether a subject has an increased likelihood of having a disorder related to abnormal intracellular calcium store discharge, the method comprising:
(a) detecting an intracellular calcium concentration F₂ in a lymphocyte obtained from the subject while the lymphocyte is in contact with an antagonist of calcium influx;
(b)(1) detecting an intracellular calcium concentration F₁ in the lymphocyte while the lymphocyte is in contact with a calcium-containing solution, and/or
(b)(2) detecting an intracellular calcium concentration F₃ in the lymphocyte after the lymphocyte has been contacted with a calcium-mobilizing agent, or before and after the lymphocyte has been contacted with the calcium-mobilizing agent;
(c)(1) determining an intracellular calcium concentration difference Δ₁, wherein Δ₁ equals F₁ minus F₂, and/or
(c)(2) determining an intracellular calcium concentration difference Δ₂, wherein Δ₂ equals F₃ minus F₂; and
(d) determining whether the subject has an increased likelihood of having a disorder related to abnormal intracellular calcium store discharge based upon the value of A1 and/or Δ₂.
2. The method of embodiment 1, wherein different lymphocytes are contacted in steps (a), (b)(1), and/or (b)(2).
3. The method of embodiment 1, wherein the same lymphocyte is contacted in all of steps (a), (b)(1), and/or (b)(2).
4. The method of embodiment 3, wherein step (b)(1) is performed before step (a).
5. The method of embodiment 3 or 4, wherein step (b)(2) is performed after step (a).
6. The method of any one of embodiments 1 to 5, wherein the lymphocyte is isolated from a sample obtained from the subject.
7. The method of embodiment 6, wherein the sample is a whole blood sample, lymph sample, tonsillar tissue sample, lung tissue sample, mucosal tissue sample, or a combination thereof.
8. The method of any one of embodiments 1 to 7, wherein the lymphocyte is a T lymphocyte.
9. The method of any one of embodiments 1 to 8, wherein the lymphocyte comprises a plurality of lymphocytes.
10. The method of any one of embodiments 1 to 9, wherein the lymphocyte is contacted with the calcium-mobilizing agent while the lymphocyte is in contact with the antagonist of calcium influx.
11. The method of any one of embodiments 1 to 10, wherein F₂ is the lowest intracellular calcium concentration detected before or after the lymphocyte is contacted with the calcium-mobilizing agent.
12. The method of any one of embodiments 1 to 11, wherein F₃ is detected between about 1 second and about 10 minutes after the lymphocyte has been contacted with the calcium-mobilizing agent.
13. The method of any one of embodiments 1 to 11, wherein F₃ is the highest intracellular calcium concentration detected after the lymphocyte has been contacted with the calcium-mobilizing agent.
14. The method of any one of embodiments 1 to 11, wherein F₃ is detected at two or more time points, wherein the earliest time point is t₁ and the latest time point is t₂.
15. The method of embodiment 14, wherein t₁ is about 5 to 10 minutes before the lymphocyte is contacted with the calcium-mobilizing agent.
16. The method of embodiment 14, wherein t₂ is about 5 to 10 minutes after the lymphocyte has been contacted with the calcium-mobilizing agent.
17. The method of any one of embodiments 1 to 16, wherein the calcium-containing solution comprises a physiological saline solution, Ringer's solution, Tyrode's solution, Hank's solution, or a combination thereof.
18. The method of any one of embodiments 1 to 17, wherein the concentration of calcium in the calcium-containing solution is between about 0.1 mM and about 100 mM.
19. The method of any one of embodiments 1 to 18, wherein the concentration of calcium in the calcium-containing solution is about 2 mM.
20. The method of any one of embodiments 1 to 19, wherein the antagonist of calcium influx comprises a calcium-free physiological saline solution, a calcium chelating agent, a calcium channel blocker, or a combination thereof.
21. The method of embodiment 20, wherein the calcium channel blocker is selected from the group consisting of a store-operated calcium channel blocker, a transient receptor potential (TRP) channel blocker, and a combination thereof.
22. The method of any one of embodiments 1 to 21, wherein the calcium-mobilizing agent is selected from the group consisting of a sarco/endoplasmic reticulum Ca²⁺-ATPase (SERCA) inhibitor, a calcium ionophore, a membrane receptor agonist, an intracellular receptor agonist, and a combination thereof.
23. The method of embodiment 22, wherein the membrane receptor agonist is a T lymphocyte receptor agonist.
24. The method of embodiment 22, wherein the intracellular receptor agonist is an inositol trisphosphate receptor (IP3R) agonist and/or a ryanodine receptor (RyR) agonist.
25. The method of any one of embodiments 1 to 24, wherein the calcium-mobilizing agent is present at a concentration of about 0.001 μM to about 10 mM.
26. The method of any one of embodiments 1 to 25, wherein the intracellular calcium concentration F₁, F₂, and/or F₃ is detected by observing or recording a signal.
27. The method of embodiment 26, wherein the signal is proportional to the intracellular calcium concentration.
28. The method of embodiment 26 or 27, wherein the signal is generated using a fluorescent calcium indicator.
29. The method of embodiment 28, wherein the lymphocyte is loaded with the fluorescent calcium indicator before being contacted with the calcium-containing solution.
30. The method of any one of embodiments 1 to 29, wherein the method further comprises contacting the lymphocyte with a lymphocyte activating agent.
31. The method of any one of embodiments 1 to 30, wherein the determination of whether the subject has an increased likelihood of having a disorder related to abnormal intracellular calcium store discharge is based upon the difference between Δ₁ and Δ₂.
32. The method of any one of embodiments 1 to 30, wherein the determination of whether the subject has an increased likelihood of having a disorder related to abnormal intracellular calcium store discharge is based upon the ratio of Δ₁ to Δ₂.
33. The method of any one of embodiments 1 to 32, wherein the value of Δ₁, Δ₂, the difference between Δ₁ and Δ₂, or the ratio of Δ₁ to Δ₂ is compared to a reference value.
34. The method of embodiment 33, wherein the reference value is determined from a control subject or a control population.
35. The method of embodiment 34, wherein the control subject or control population is a subject or a population of subjects who do not have a disorder related to abnormal intracellular calcium store discharge.
36. The method of any one of embodiments 1 to 35, wherein the disorder related to abnormal intracellular calcium store discharge is selected from the group consisting of Alzheimer's disease (AD), Parkinson's disease, Huntington's disease, malignant hyperthermia (MH), central core disease, multi-minicore disease (MmD), nemaline rod myopathy (NM), a T lymphocyte-mediated autoimmune disease, and exposure to a chemical pollutant.
37. The method of embodiment 36, wherein the chemical pollutant is selected from the group consisting of a polychlorinated biphenyl compound, triclosan, bisphenol A, and a combination thereof.
38. The method of embodiment 36, wherein the T lymphocyte-mediated autoimmune disease is selected from the group consisting of multiple sclerosis (MS), myasthenia gravis, an autoimmune neuropathy, uveitis, autoimmune hemolytic anemia, pernicious anemia, autoimmune thrombocytopenia, temporal arteritis, anti-phospholipid syndrome, autoimmune vasculitis, Bechet's disease, atherosclerosis, psoriasis, dermatitis herpetiformis, pemphigus vulgaris, vitiligo, mycosis fungoides, allergic contact dermatitis, atopic dermatitis, lichen planus, pityriasis lichenoides at varioliforms acute (PLEVA), Crohn's disease, ulcerative colitis, primary biliary cirrhosis, autoimmune hepatitis, type I diabetes mellitus, Addison's disease, Grave's disease, and Hashimoto's thyroiditis.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

What is claimed is:

1. A method for determining whether a subject has an increased likelihood of having a disorder related to abnormal intracellular calcium store discharge, the method comprising:
(a) detecting an intracellular calcium concentration F₂ in a lymphocyte obtained from the subject while the lymphocyte is in contact with an antagonist of calcium influx;
(b)(1) detecting an intracellular calcium concentration F₁ in the lymphocyte while the lymphocyte is in contact with a calcium-containing solution, and/or
(b)(2) detecting an intracellular calcium concentration F₃ in the lymphocyte after the lymphocyte has been contacted with a calcium-mobilizing agent, or before and after the lymphocyte has been contacted with the calcium-mobilizing agent;
(c)(1) determining an intracellular calcium concentration difference Δ₁, wherein Δ₁ equals F₁ minus F₂, and/or
(c)(2) determining an intracellular calcium concentration difference Δ₂, wherein Δ₂ equals F₃ minus F₂; and
(d) determining whether the subject has an increased likelihood of having a disorder related to abnormal intracellular calcium store discharge based upon the value of Δ₁ and/or Δ₂.

2. The method of claim 1, wherein different lymphocytes are contacted in steps (a), (b)(1), and/or (b)(2).

3. The method of claim 1, wherein the same lymphocyte is contacted in all of steps (a), (b)(1), and/or (b)(2).

4. The method of claim 3, wherein step (b)(1) is performed before step (a).

5. The method of claim 3, wherein step (b)(2) is performed after step (a).

6. The method of claim 1, wherein the lymphocyte is isolated from a sample obtained from the subject.

7. The method of claim 6, wherein the sample is a whole blood sample, lymph sample, tonsillar tissue sample, lung tissue sample, mucosal tissue sample, or a combination thereof.

8. The method of claim 1, wherein the lymphocyte is a T lymphocyte.

9. The method of claim 1, wherein the lymphocyte comprises a plurality of lymphocytes.

10. The method of claim 1, wherein the lymphocyte is contacted with the calcium-mobilizing agent while the lymphocyte is in contact with the antagonist of calcium influx.

11. The method of claim 1, wherein F₂ is the lowest intracellular calcium concentration detected before or after the lymphocyte is contacted with the calcium-mobilizing agent.

12. The method of claim 1, wherein F₃ is detected between about 1 second and about 10 minutes after the lymphocyte has been contacted with the calcium-mobilizing agent.

13. The method of claim 1, wherein F₃ is the highest intracellular calcium concentration detected after the lymphocyte has been contacted with the calcium-mobilizing agent.

14. The method of claim 1, wherein F₃ is detected at two or more time points, wherein the earliest time point is t₁ and the latest time point is t₂.

15. The method of claim 14, wherein t₁ is about 5 to 10 minutes before the lymphocyte is contacted with the calcium-mobilizing agent.

16. The method of claim 14, wherein t₂ is about 5 to 10 minutes after the lymphocyte has been contacted with the calcium-mobilizing agent.

17. The method of claim 1, wherein the calcium-containing solution comprises a physiological saline solution, Ringer's solution, Tyrode's solution, Hank's solution, or a combination thereof.

18. The method of claim 1, wherein the concentration of calcium in the calcium-containing solution is between about 0.1 mM and about 100 mM.

19. The method of claim 1, wherein the concentration of calcium in the calcium-containing solution is about 2 mM.

20. The method of claim 1, wherein the antagonist of calcium influx comprises a calcium-free physiological saline solution, a calcium chelating agent, a calcium channel blocker, or a combination thereof.

21. The method of claim 20, wherein the calcium channel blocker is selected from the group consisting of a store-operated calcium channel blocker, a transient receptor potential (TRP) channel blocker, and a combination thereof.

22. The method of claim 1, wherein the calcium-mobilizing agent is selected from the group consisting of a sarco/endoplasmic reticulum Ca²⁺-ATPase (SERCA) inhibitor, a calcium ionophore, a membrane receptor agonist, an intracellular receptor agonist, and a combination thereof.

23. The method of claim 22, wherein the membrane receptor agonist is a T lymphocyte receptor agonist.

24. The method of claim 22, wherein the intracellular receptor agonist is an inositol trisphosphate receptor (IP3R) agonist and/or a ryanodine receptor (RyR) agonist.

25. The method of claim 1, wherein the calcium-mobilizing agent is present at a concentration of about 0.001 μM to about 10 mM.

26. The method of claim 1, wherein the intracellular calcium concentration F₁, F₂, and/or F₃ is detected by observing or recording a signal.

27. The method of claim 26, wherein the signal is proportional to the intracellular calcium concentration.

28. The method of claim 26, wherein the signal is generated using a fluorescent calcium indicator.

29. The method of claim 28, wherein the lymphocyte is loaded with the fluorescent calcium indicator before being contacted with the calcium-containing solution.

30. The method of claim 1, wherein the method further comprises contacting the lymphocyte with a lymphocyte activating agent.

31. The method of claim 1, wherein the determination of whether the subject has an increased likelihood of having a disorder related to abnormal intracellular calcium store discharge is based upon the difference between Δ₁ and Δ₂.

32. The method of claim 1, wherein the determination of whether the subject has an increased likelihood of having a disorder related to abnormal intracellular calcium store discharge is based upon the ratio of Δ₁ to Δ₂.

33. The method of claim 1, wherein the value of Δ₁, Δ₂, the difference between Δ₁ and Δ₂, or the ratio of Δ₁ to Δ₂ is compared to a reference value.

34. The method of claim 33, wherein the reference value is determined from a control subject or a control population.

35. The method of claim 34, wherein the control subject or control population is a subject or a population of subjects who do not have a disorder related to abnormal intracellular calcium store discharge.

36. The method of claim 1, wherein the disorder related to abnormal intracellular calcium store discharge is selected from the group consisting of Alzheimer's disease (AD), Parkinson's disease, Huntington's disease, malignant hyperthermia (MH), central core disease, multi-minicore disease (MmD), nemaline rod myopathy (NM), a T lymphocyte-mediated autoimmune disease, and exposure to a chemical pollutant.

37. The method of claim 36, wherein the chemical pollutant is selected from the group consisting of a polychlorinated biphenyl compound, triclosan, bisphenol A, and a combination thereof.

38. The method of claim 36, wherein the T lymphocyte-mediated autoimmune disease is selected from the group consisting of multiple sclerosis (MS), myasthenia gravis, an autoimmune neuropathy, uveitis, autoimmune hemolytic anemia, pernicious anemia, autoimmune thrombocytopenia, temporal arteritis, anti-phospholipid syndrome, autoimmune vasculitis, Bechet's disease, atherosclerosis, psoriasis, dermatitis herpetiformis, pemphigus vulgaris, vitiligo, mycosis fungoides, allergic contact dermatitis, atopic dermatitis, lichen planus, pityriasis lichenoides at varioliforms acute (PLEVA), Crohn's disease, ulcerative colitis, primary biliary cirrhosis, autoimmune hepatitis, type I diabetes mellitus, Addison's disease, Grave's disease, and Hashimoto's thyroiditis.
